(19) United States
(12) Patent Application Publication
(10) Pub. No.: US 2015/0047664 A1
(43) Pub. Date: Feb. 19, 2015

(54) COSMETIC PROCESS FOR CARING FOR AND/OR MAKING UP KERATIN MATERIALS

(71) Applicant: L'OREAL, Paris (FR)

(72) Inventors: Henri Samain, Bievres (FR); Gregory Plos, Paris (FR); Anne Bouchara, Paris (FR)

(73) Assignee: L'OREAL, Paris (FR)

(21) Appl. No.: 14/387,667

(22) PCT Filed: Mar. 27, 2013
(86) PCT No.: PCT/IB13/52447

§ 371 Date: Sep. 24, 2014

Related U.S. Application Data
(60) Provisional application No. 61/709,198, filed on Oct. 3, 2012.

(30) Foreign Application Priority Data
Mar. 27, 2012 (FR) 1252729

(51) Int. Cl. A61K 8/58; A61Q 3/02; A61K 8/19; A61K 8/43; A61K 8/365; A61Q 5/00; A61K 8/46

(52) U.S. Cl. CPC A61K 8/585; A61Q 5/00; A61Q 3/02; A61K 8/46; A61K 8/43; A61K 8/365; A61K 8/19
USPC 132/202; 424/70.1; 424/61; 132/200

(57) ABSTRACT

Cosmetic process for caring for and/or making up keratin materials The present invention relates to a cosmetic process for caring for and/or making up keratin materials, comprising at least the steps consisting in: (i) placing all or part of the surface of the said keratin material in contact with an effective amount of at least one emollient active agent chosen from ionic liquids based on guanidinium or dialkylimidazolium, non-volatile or sparingly volatile oils, waxes, thiol derivatives, phosphines, alkaline bases chosen from potassium hydroxide, ammonia, monoethanolamine, triethanolamine, calcium hydroxide, alkaline salts of amino acids, urea and urea derivatives, and guanidine derivatives, and mixtures thereof, (ii) placing the said surface in contact with at least one cosmetic active agent, different from the said emollient active agent, to be incorporated into the keratin at the surface of the said keratin material, and (iii) where appropriate, heating the said surface of the keratin material, steps (ii) and (iii) possibly being performed, independently of each other, prior to, simultaneously with or consecutive to step (i).

The present invention relates to a cosmetic process for caring for and/or making up keratin materials, which is in particular directed towards incorporating into the keratin forming the said keratin materials a cosmetic active agent, for instance a solid and/or hydrophobic material.

In particular, the present invention relates to a process for making up and/or caring for the hair and/or the nails, more particularly the nails, in order in particular to reinforce the keratin of which they are constituted.

Keratin is a basic structural element of keratin materials and is particularly preponderant in keratin materials such as the hair and the nails. It especially provides a large part of their mechanical properties such as their tensile strength, their breaking load and their elasticity. Thus, keratin generally has a hardness characterized by a Young's modulus of about 3 GPa.

However, it is known that keratin materials are sensitized, i.e. damaged and/or embrittled, on a daily basis to varying degrees by the action of atmospheric agents, especially light, and also by the repeated action of various mechanical or chemical treatments. These attacking factors have the effect of reducing their mechanical properties.

Consequently, it would, in certain circumstances, be advantageous or even necessary to be able to modify the surface properties of keratin materials in order notably to reinforce their mechanical properties, but also to give them novel properties, for example a repellent effect with regard to water, or increased impermeability with regard to fatty substances.

Modification of the surface properties, and in particular the mechanical properties, of a keratin material may require the incorporation of active agent(s) into the keratin forming this keratin material, with the difficulties then firstly of precisely enabling these active agents to overcome the mechanical barrier naturally constituted by keratin and secondly of incorporating them or encrusting them into the keratin in an amount sufficient to draw benefit therefrom.

Various processes for incorporating active agents into keratin have already been proposed.

A first technique, more particularly considered for reinforcing the mechanical properties of a keratin material such as the nails or the hair, is directed towards introducing hard materials directly into the keratin. However, it is certain that a hard material can only have an effect on the mechanical strength if the amount introduced is large. Now, it is precisely difficult to incorporate large amounts with regard to the native mechanical properties of keratin.

Silicon-bearing materials of alkoxysilane type, in particular with (3-aminopropyl)triethoxysilane (APTES), have already been proposed for these purposes. However, their satisfactory performance, in terms of penetration and hardness, have an excessively limited duration over time.

Other techniques are based on the use of crosslinking agents. However these agents are not always entirely satisfactory since they often involve compounds with an adverse side effect.

Finally, other approaches, based on the grafting or insertion of an active agent into the disulfide bridges of keratin, have also been proposed for the purposes of consolidating the mechanical properties of keratin fibres. However, they do not afford significant reinforcing effects.

There is consequently a need for cosmetic processes for modifying the surface properties of keratin materials, in particular for reinforcing their mechanical properties such as the tensile strength, the breaking load and the elasticity in a durable and significant manner, and which do not have any adverse side effects.

Contrary to all expectation, the inventors have found that the above-mentioned advantages can be achieved provided that a specific treatment of the keratin material under consideration is performed in conjunction with the step of incorporating the material under consideration.

Thus, according to one of its aspects, one subject of the present invention is a cosmetic process for caring for and/or making up keratin materials, comprising at least the steps consisting in:

(i) placing all or part of the surface of the said keratin material in contact with an effective amount of at least one emollient active agent chosen from ionic liquids, non-volatile or sparingly volatile oils, waxes, thiol derivatives, phosphines, acids and alkaline bases, and mixtures thereof,

(ii) placing the said surface in contact with at least one cosmetic active agent, different from the said emollient active agent, to be incorporated into the keratin at the surface of the said keratin material, and

(iii) where appropriate, heating the said surface of the keratin material, steps (ii) and (iii) possibly being performed, independently of each other, prior to, simultaneously with or consecutive to step (i).

More particularly, the present invention relates to a cosmetic process for caring for and/or making up keratin materials, comprising at least the steps consisting in:

(i) placing all or part of the surface of the said keratin material in contact with an effective amount of at least one emollient active agent chosen from ionic liquids based on guanidinium or dialkylimidazolium, non-volatile or sparingly volatile oils, waxes, thiol derivatives, phosphines, alkaline bases chosen from potassium hydroxide, ammonia, monoethanolamine, triethanolamine, calcium hydroxide, alkaline salts of amino acids, urea and urea derivatives, and guanidine derivatives, and mixtures thereof,

(ii) placing the said surface in contact with at least one cosmetic active agent, different from the said emollient active agent, to be incorporated into the keratin at the surface of the said keratin material, and

(iii) where appropriate, heating the said surface of the keratin material, steps (ii) and (iii) possibly being performed, independently of each other, prior to, simultaneously with or consecutive to step (i).

According to a preferred variant, step (iii), if it exists, is performed prior to or simultaneously with step (i).

More particularly, the emollient active agent(s) under consideration in step (i) are capable of reducing the Young's modulus (GpA) of the said keratin material by at least a factor of 4.

According to a preferred embodiment, the emollient active agent is chosen from ionic liquids based on guanidinium or dialkylimidazolium, cysteine or thioglycolic acid, and guanidine derivatives, and mixtures thereof.

Preferably, the emollient active agent is a thiol derivative or a phosphine combined with a guanidinium-based ionic liquid.

As emerges from the text hereinbelow, the step of softening keratin proves to be particularly effective for promoting significant encrustation of the cosmetic active agent therein.

In the case where the two steps are performed in conjunction, the application of the two compositions containing, respectively, the emollient active agent and the active agent to be encrusted or of a composition resulting from the mixing at the time of use of the two types of active agent may be performed directly on the keratin material.

If a single composition is applied, it will have the capacity of performing the two steps (i) and (ii) as described above.

For the purposes of the present invention, the term “keratin materials” is intended to cover the skin, mucous membranes such as the lips, the nails and keratin fibres, such as the eyelashes and the hair. The hair and the nails, and in particular the nails, are most particularly considered according to the invention.

For the purposes of the invention, the cosmetic active agent to be incorporated into the keratin of the said keratin material may or may not be solid.

According to one embodiment variant, the cosmetic active agent to be incorporated into keratin may be used in a form dissolved in a formulation medium.

According to one embodiment variant, the cosmetic active agent may be a reactive compound that is in particular capable of reacting with nucleophiles such as formaldehyde, aldehydes, di- or poly-aldehydes, aldehydes or ketones, or esters or organic anhydrides.

According to one embodiment variant, this cosmetic active agent may be of mineral or organic nature.

Thus, this cosmetic active agent may be a mineral particulate material such as pigments, which are advantageous with regard to the optical effect they afford, for example a whitening or lightening effect on the keratin material incorporating them, or alternatively silica particles, which are advantageous for reinforcing the mechanical properties of a keratin material.

According to another embodiment variant, the active agent is organic and may be of monomeric, oligomeric or polymeric nature, and in particular hydrophilic or hydrophobic.

An active agent of elastomeric or polymeric nature may in particular be featured by an acrylic, methacrylic, polyurethane, polyester, polyamide or polyholoside copolymer or derivatives thereof. It may also be an organic active agent which, after transformation, gives a polymeric material, for example a reactive silicone. These active agents are especially advantageous for imparting, via their incorporation into keratin at the surface of the said keratin material, attachment properties with regard to a surface treatment following, for example, the application of a film of varnish type in the case of a keratin material such as the nails or alternatively of a fixing film of lacquer type in the case of a keratin material such as the hair.

For its part, an active agent of elastomeric or polymeric nature with hydrophobic character, for instance an aromatic compound, a perfluoro compound or a wax, may be advantageous for imparting, via its incorporation into keratin at the surface of the said keratin material, hydrophobic properties.

According to a preferred embodiment variant, this cosmetic active agent is a hardener or reinforcer and, in this respect, is advantageous for consolidating the mechanical properties of a keratin material.

For the purposes of the invention, an active agent is termed “hardening” or “reinforcing” with regard either to its capacity for reinforcing the mechanical properties of a keratin material by means of its incorporation as such into keratin at the surface of the said keratin material, like, for example, mineral particles in particular such as silica or alkoxysilane derivatives as described below, or to its capacity for forming in situ in keratin solid particles, like, for example, a siccative oil, or to its capacity for generating in situ a composite via its incorporation with the keratin incorporating it or via its own self-condensation.

In this second alternative, the hardening compound condenses onto itself without appreciable reaction with the functions of keratin, like, for example, silanes bearing a reactive function, aldehydes or ketones. In this second alternative, the hardening compound may also be a crosslinking agent which reacts on the functions of keratin, like, for example, a di- or multi-reactive compound reacting on the mercaptan functions of keratin or on the amine functions of keratin, such as dithiols or dialdehydes.

For obvious reasons, a single cosmetic active agent as described above is capable of ensuring, by means of its presence in keratin, several functions, for example a hardening function in conjunction with a lightening effect in the case of certain pigments.

Similarly, the process according to the invention is compatible with the simultaneous or consecutive incorporation of two different cosmetic active agents that afford different effects, for example silica particles affording a hardening effect and an elastomeric material affording a hydrophobic property.

According to one of its aspects, a subject of the invention is also a cosmetic care and/or makeup process for the hardening or reinforcing, in terms of thickness, of keratin materials, especially the nails or keratin fibres, in particular the hair, comprising at least the steps consisting in:

(i) placing all or part of the surface of the said keratin material in contact with an effective amount of at least one emollient active agent, which is capable of reducing by at least a factor of 4 the native Young's modulus (GpA) of the said keratin material; and

(ii) placing the said surface thus softened in contact with at least one hardening or reinforcing active agent, different from the said emollient active agent, to be incorporated into the keratin at the surface of the said keratin material;

(iii) where appropriate, heating the said surface of the keratin material,

steps (ii) and (iii) possibly being performed, independently of each other, prior to, simultaneously with or consecutive to step (i).

According to a preferred embodiment, the emollient active agent is chosen from ionic liquids based on guanidinium or dialkylimidazolium, non-volatile or sparingly volatile oils, waxes, thiol derivatives, in particular cysteine, phosphines, alkaline bases chosen from potassium hydroxide, ammonia, monoethanolamine, triethanolamine, calcium hydroxide, alkaline salts of amino acids, in particular glycinate salts, urea and urea derivatives, guanidine derivatives, in particular guanidine hydroxide, and guanidine carbonate, and more particularly guanidine carbonate, and mixtures thereof.

According to a preferred variant, step (iii), if it exists, is performed prior to or simultaneously with step (i).

In this embodiment variant, the emollient active agent is advantageously chosen from ionic liquids, non-volatile or sparingly volatile oils, waxes, thiol derivatives, phosphines, acids and alkaline bases, and mixtures thereof.

In this embodiment variant, the hardener or reinforcer is more particularly chosen from an alkoxysilane compound, a siccative oil, a ceramide, a polymer and/or a crosslinking agent.

According to another of its aspects, a subject of the invention is a cosmetic care and/or makeup process for hardening the nails, comprising at least the steps consisting in:

(i) placing all or part of the surface of the said nail in contact with an effective amount of at least one emollient active agent chosen from ionic liquids and thiol derivatives, and mixtures thereof,

(ii) placing the said surface thus softened in contact with an alkoxysilane compound chosen from (3-aminopropyl)triethoxysilane (APTES), methyltriethoxysilane (MTES) and octyltriethoxysilane (OTES), to be incorporated into the keratin at the surface of the said nail,

(iii) where appropriate, heating the said surface of the nail,

steps (ii) and (iii) possibly being performed, independently of each other, prior to, simultaneously with or consecutive to step (i).

In particular, the present invention relates to a cosmetic care and/or makeup process for hardening the nails, comprising at least steps consisting in:

(i) placing all or part of the surface of the said nail in contact with an effective amount of at least one emollient active agent chosen from ionic liquids based on guanidinium or dialkylimidazolium, thiol derivatives, bases chosen from potassium hydroxide, ammonia, monoethanolamine, triethanolamine, calcium hydroxide, alkaline salts of amino acids, urea and urea derivatives, and guanidine derivatives, and mixtures thereof,

(ii) placing the said surface thus softened in contact with an alkoxysilane compound chosen from (3-aminopropyl)triethoxysilane (APTES), methyltriethoxysilane

(MTES) and octyltriethoxysilane (OTES), to be incorporated into the keratin at the surface of the said nail, and

(iii) where appropriate, heating the said surface of the nail,

steps (ii) and (iii) possibly being performed, independently of each other, prior to, simultaneously with or consecutive to step (i).

Emollient Active Agent

For the purposes of the present invention, an emollient active agent is a compound or material that is capable of significantly lowering the Young's modulus characterizing the hardness of a keratin material.

The Young's modulus characterizes the strength of a material exposed to a mechanical action. Thus, it characterizes the force to the imposed, per unit of surface area, to produce a change in the material.

The emollience, afforded by step (i), takes place mainly on the surface of the keratin material, it being understood that it is not essential for it to take place throughout the entire depth of the keratin material.

More precisely, the softening of the keratin material thus obtained does not extend deep down.

Preferably, the softening of the keratin material does not extend beyond a depth of 10 micrometres.

The measuring method for characterizing step (i) is a method of microindentation with an indentation depth of at least 2 micrometres. Thus, according to the invention, the Young's modulus may be advantageously reduced by a factor of 4 relative to a native Young's modulus of the surface of the keratin material.

Step (i) may also be characterized by atomic force microscopy (AFM). In “tapping” mode, access to the level of viscoelasticity of the surface is gained without indication of the depth. In comparison with an untreated control hair, a change in viscoelasticity may thus be revealed.

As stated previously, the active agent under consideration for affording such a softening of keratin at the surface of the keratin material under consideration is chosen from ionic liquids, non-volatile or sparingly volatile oils, waxes, thiol derivatives, phosphines, acids and alkaline bases, and mixtures thereof.

In one embodiment variant, the emollient active agent may consist of a single compound, for example a single ionic liquid, or may be formed from a mixture of compounds of the same chemical nature, for example a mixture of two, or even more, ionic liquids.

In another embodiment variant, the emollient active agent may be formed from emollient compounds of different chemical nature. For example, an emollient active agent according to the invention may consist of a mixture of at least one ionic liquid with a thiol derivative.

In another embodiment variant, the emolliation may consist of the successive application of at least two different emollient active agents.

As emerges from the text hereinbelow, the use of the emollient active agent(s) may, where appropriate, be subordinate to particular application conditions, for instance heating, optionally concomitantly, of the said keratin material and/or of the said active agent.

The heating may, for example, be performed by exposure to UV and, as more particularly regards keratin fibres, the passage of flat tongs over the hair, such as a styling iron, or the passage of a hairdryer.

Such heating proves to be most particularly advantageous in the case of certain active agents either for stimulating their emollient effect with regard to the keratin material, or for compensating for an excessively prolonged action time of certain active agents at room temperature. Included in the first category are, for example, thiols or alkaline agents, and in the second category are ionic liquids or oils.

Thus, hair treated with an emollient active agent such as, for example, an oil, an ionic liquid or a mixture thereof will also be heated via a standard heating device, for instance a hairdryer or a styling iron at a temperature of 60° C., or even of 100° C.

Needless to say, these particular conditions are to be adjusted in order to be compatible with application to the keratin material under consideration. For obvious reasons, these adjustments fall within the competence of a person skilled in the art.

### a) Ionic Liquid

For the purposes of the present invention, the term “ionic liquid” means a salt of an organic molecule, the said salt having a melting point of less than or equal to 150° C. and preferably less than 100° C.

Preferably, the salt remains liquid up to 300° C., and more preferentially the salt is liquid at room temperature, i.e. at a temperature of less than or equal to 50° C. and greater than 0° C.

The melting point is measured by differential calorimetric analysis, with a temperature increase rate of 10° C./minute, the melting point then being at a temperature corresponding to the top of the melting endotherm peak obtained during the measurement.

The salt may be derived from the combination of a mineral or organic anion and a mineral or organic cation. Preferably, the organic molecule constitutes the cation and the anion may be mineral or organic.

The ionic liquid(s) used according to the invention advantageously have a mineral or organic cation preferably chosen from imidazolium, pyrazolium, pyridinium, pyrimidinium, tetra(C₁-C₆)alkylphosphonium, tetra(C₁-C₆)alkylammonium, guanidinium, cholinium, pyrrolidinium, uronium, thiouronium and isothiouronium cations.

Preferably, the cation of the ionic liquid(s) used according to the invention is chosen from guanidinium, ammonium, imidazolium and cholinium cations.

Preferably, the anion of the ionic liquid(s) used according to the invention is chosen from acetate, acetate derivatives, propionate, carbonate, chloride, hydroxide, sulfate, sulfate derivatives and phosphates.

In a non-exhaustive manner, the ionic liquid(s) used according to the invention may be chosen from the following compounds: 1-ethyl-3-methylimidazolium chloride, 1-ethyl-3-methylimidazolium bromide, 1-butyl-3-methylimidazolium chloride, 1-hexyl-3-methylimidazolium chloride, 1-methyl-3-octylimidazolium chloride, 1-decyl-3-methylimidazolium chloride, 1-decyl-3-methylimidazolium bromide, 1-dodecyl-3-methylimidazolium chloride, 1-methyl-3-tetradecylimidazolium chloride, 4-methyl-N-butylpyridinium chloride, 3-methyl-N-butylpyridinium chloride, 4-methyl-N-hexylpyridinium chloride, 1-ethyl-3-methylimidazolium tetrafluoroborate, 1-butyl-3-methylimidazolium tetrafluoroborate, 1-pentyl-3-methylimidazolium tetrafluoroborate, 1-hexyl-3-methylimidazolium tetrafluoroborate, 1-heptyl-3-methylimidazolium tetrafluoroborate, 1-octyl-3-methylimidazolium tetrafluoroborate, 1-nonyl-3-methylimidazolium tetrafluoroborate, 1-decyl-3-methylimidazolium tetrafluoroborate, 4-methyl-N-butylpyridinium tetrafluoroborate, 1-hexyl-3-ethylimidazolium tetrafluoroborate, 1-ethyl-3-methylimidazolium hexafluorophosphate, 1-butyl-3-methylimidazolium hexafluorophosphate, 1-pentyl-3-methylimidazolium hexafluorophosphate, 1-hexyl-3-methylimidazolium hexafluorophosphate, 1-heptyl-3-methylimidazolium hexafluorophosphate, 1-octyl-3-methylimidazolium hexafluorophosphate, 1-nonyl-3-methylimidazolium hexafluorophosphate, 1-decyl-3-methylimidazolium hexafluorophosphate, 1,3-dimethylimidazolium methyl sulfate, 1-methyl-3-butylimidazolium methyl sulfate, 1-ethyl-3-methylimidazolium nitrate, 1-ethyl-3-methylimidazolium nitrite, 1-ethyl-3-methylimidazolium acetate, 1-ethyl-3-methylimidazolium sulfate, 1-ethyl-3-methylimidazolium triflates, 1-ethyl-3-methylimidazolium nonaflates, 1-ethyl-3-methylimidazolium bis(trityl)amide, 1-butylpyridinium bromide, 1-butylpyrimidinium trifluoromethanesulfonate, 1-hexylpyrimidinium trifluoromethanesulfonate, 1-ethyl-3-methylimidazolium trifluoroacetate, trihexyltetradecylphosphonium chloride, tributyltetradecylphosphonium chloride, 1-ethyl-2-methylpyrazolium tetrafluoroborate, 1-methyl-3-butylpyrimidinium tetrafluoroborate, and 1-ethyl-3-methylimidazolium trifluoroacetate, 1-hexyl-2,3-dimethylimidazolium chloride, guanidinium tris(pentafluoroethyl)trifluorophosphate, guanidinium carbonate, 1-ethyl-2,3-dimethylimidazolium chloride, 1-ethyl-3-methylimidazolium dicyanamide, 1-butyl-3-methylimidazolium dicyanamide, tetrabutylammonium hydroxide, choline salicylate, tributylmethylammonium methyl sulfate, cholinium acetate, tetraethylammonium acetate tetrahydrate, triethylmethylammonium dibutyl phosphate, 1-ethyl-3-methylimidazolium L-(+)-lactate, and hydrates thereof.

Preferentially, the invention uses as emollient active agent at least one ionic liquid based on guanidinium or dialkylimidazolium.

Preferably, the emollient active agent is at least based on dialkylimidazolium, preferentially an ionic liquid based on a dialkylimidazolium acetate and in particular 1-ethyl-3-methylimidazolium acetate or chloride.

More preferentially, the emollient active agent is at least 1-ethyl-3-methylimidazolium acetate. Such an active agent proves to be most particularly advantageous with regard to keratin fibres and more particularly the hair.

In general, the ionic liquid or the mixture of ionic liquids is used in a proportion of from 1% to 100% by weight and in particular from 20% to 90% by weight of a composition containing it.

Preferably, when the ionic liquid is not used per se, it is formulated with a polar solvent, and preferably water.

### b) Non-Volatile or Sparingly Volatile Oil

According to an advantageous embodiment, the emollient active agent according to the invention may be at least one non-volatile or sparingly volatile oil.

The term “oil” means a water-immiscible non-aqueous compound that is liquid at room temperature (25° C.) and at atmospheric pressure (760 mmHg).

The term “non-volatile” oil refers to an oil whose vapour pressure at room temperature and atmospheric pressure is non-zero and less than 0.02 mmHg (2.66 Pa) and better still less than 10⁻³ mmHg (0.13 Pa).

The term “sparingly volatile” oil refers to an oil whose vapour pressure at room temperature and atmospheric pressure is greater than 0.02 mmHg (2.66 Pa) and less than 0.08 mmHg (10.6 Pa) and better still greater than 0.04 mmHg (5.3 Pa) and less than 0.06 mmHg (8 Pa).

The non-volatile or sparingly volatile oils may be hydrocarbon-based oils in particular of plant origin, oils of synthetic or mineral origin, silicone oils, fluoro oils, or mixtures thereof.

Apolar Oil

According to a first embodiment, the said non-volatile or sparingly volatile oil may be an apolar oil, preferably an apolar hydrocarbon-based oil.

These oils may be of plant, mineral or synthetic origin. For the purposes of the present invention, the term “apolar oil” means an oil whose solubility parameter at 25° C., δₐ, is equal to 0 (J/cm³)^{1/2}.

The definition and calculation of the solubility parameters in the Hansen three-dimensional solubility space are described in the article by C. M. Hansen: The three dimensional solubility parameters, J. Paint Technol., 39, 105 (1967).

According to this Hansen space:
δ_{D} characterizes the London dispersion forces derived from the formation of dipoles induced during molecular impacts;
δₚ characterizes the Debye interaction forces between permanent dipoles and also the Keesom interaction forces between induced dipoles and permanent dipoles;
δₕ characterizes the specific interaction forces (such as hydrogen bonding, acid/base, donor/acceptor, etc.); and
δₐ is determined by the equation: δₐ=(δₚ²+δₕ²)^{1/2}.

The parameters δₚ, δₕ, δ_{D} and δₐ are expressed in (J/cm³)^{1/2}.

The term “hydrocarbon-based oil” means an oil formed essentially from, indeed even consisting of, carbon and hydrogen atoms, and optionally oxygen and nitrogen atoms, and not containing any silicon or fluorine atoms. It may contain alcohol, ester, ether, carboxylic acid, amine and/or amide groups.

Preferably, the non-volatile apolar hydrocarbon-based oil may be chosen from linear or branched hydrocarbons of mineral or synthetic origin, such as:
liquid paraffin or derivatives thereof,
petrolatum,
squalane,
isoeicosane,
naphthalene oil,
polybutylenes such as Indopol H-100 (molar mass or MW=965 g/mol), Indopol H-300 (MW=1340 g/mol) and Indopol H-1500 (MW=2160 g/mol) sold or manufactured by the company Amoco,
hydrogenated polyisobutylenes such as Parleam® sold by the company Nippon Oil Fats Corporation, Panalane H-300 E sold or manufactured by the company Amoco (MW=1340 g/mol), Viseal 20000 sold or manufactured by the company Synteal (MW=6000 g/mol) or Rewopal PIB 1000 sold or manufactured by the company Witco (MW=1000 g/mol),
decene/butene copolymers, polybutene/polyisobutene copolymers, especially Indopol L-14,
polydecenes and hydrogenated polydecenes such as: Puresyn 10 (MW=723 g/mol) and Puresyn 150 (MW=9200 g/mol) sold or manufactured by the company Mobil Chemicals,
and mixtures thereof.

Polar Oil

According to one particular embodiment, the emollient active agent is at least one polar oil.

Within the meaning of the present invention, the term “polar oil” means an oil for which the solubility parameter at 25° C., δₐ, is other than 0 (J/cm³)^{1/2}.

The polar oil may be a hydrocarbon-based, silicone and/or fluoro oil.

These oils may be of plant, mineral or synthetic origin.

The term “polar hydrocarbon-based oil” means an oil formed essentially from, or even consisting of, carbon and hydrogen atoms, and optionally oxygen and nitrogen atoms, and not containing any silicon or fluorine atoms. It may contain alcohol, ester, ether, carboxylic acid, amine and/or amide groups.

The term “silicone oil” means an oil containing at least one silicon atom, and especially containing Si—O groups.

The term “fluoro oil” means an oil containing at least one fluorine atom.

In particular, the polar oil may be chosen from the list of oils below, and mixtures thereof:
hydrocarbon-based polar oils such as phytostearyl esters, such as phytostearyl oleate, phytostearyl isostearate and lauroyl/octyldodecyl/phytostearyl glutamate (Ajinomoto, Eldew PS203), triglycerides consisting of fatty acid esters of glycerol, in particular the fatty acids of which may have chain lengths ranging from C₄ to C₃₆, and especially from C₁₈ to C₃₆, these oils possibly being linear or branched, and saturated or unsaturated; these oils may especially be heptanoic or octanoic triglycerides, wheatgerm oil, sunflower oil, grapeseed oil, sesame seed oil (820.6 g/mol), corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin oil, marrow oil, blackcurrant oil, evening primrose oil, millet oil, barley oil, quinoa oil, rye oil, safflower oil, candlenut oil, passionflower oil or musk rose oil; shea butter; or alternatively caprylic/capric acid triglycerides, for instance those sold by the company Stéarineries Dubois or those sold under the names Miglyol 810®, 812® and 818® by the company Dynamit Nobel;
synthetic ethers containing from 10 to 40 carbon atoms, such as dicaprylyl ether;
hydrocarbon-based esters of formula RCOOR′ in which RCOO represents a carboxylic acid residue comprising from 2 to 40 carbon atoms, and R′ represents a hydrocarbon-based chain containing from 1 to 40 carbon atoms, such as cetostearyl octanoate, isopropyl alcohol esters, such as isopropyl myristate or isopropyl palmitate, ethyl palmitate, 2-ethylhexyl palmitate, isopropyl stearate or isostearate, isostearyl isostearate, octyl stearate, diisopropyl adipate, heptanoates, and especially isostearyl heptanoate, alcohol or polyalcohol octanoates, decanoates or ricinoleates, for instance propylene glycol dioctanoate, cetyl octanoate, tridecyl octanoate, 2-ethylhexyl 4-diheptanoate and palmitate, alkyl benzoate, polyethylene glycol diheptanoate, propylene glycol 2-diethyl hexanoate, and mixtures thereof, C₁₂ to C₁₅ alcohol benzoates, hexyl laurate, neopentanoic acid esters, for instance isodecyl neopentanoate, isotridecyl neopentanoate, isostearyl neopentanoate and 2-octyldodecyl neopentanoate, isononanoic acid esters, for instance isononyl isononanoate, isotridecyl isononanoate and octyl isononanoate, oleyl erucate, isopropyl lauroyl sarcosinate, diisopropyl sebacate, isocetyl stearate, isodecyl neopentanoate, isostearyl behenate, and myristyl myristate;
polyesters obtained by condensation of an unsaturated fatty acid dimer and/or trimer and of diol, such as those described in patent application FR 0 853 634, in particular such as dilinoleic acid and 1,4-butanediol. Mention may especially be made in this respect of the polymer sold by Biosynthis under the name Viscoplast® 14436H (INCI name: dilinoleic acid/butanediol copolymer), or else copolymers of polyols and of dimer diacids, and esters thereof, such as Hailuscent ISDA®;
polyol esters and pentaerythritol esters, for instance dipentaerythrityl tetrahydroxystearate/tetraisostearate;
C₁₂-C₂₂ higher fatty acids, such as oleic acid, linoleic acid and linolenic acid, and mixtures thereof;
fluorinated oils which are optionally partially hydrocarbon-based and/or silicone-based;
silicone oils such as phenyl silicones, for instance Belsil® PDM 1000 from the company Wacker (MW=9000 g/mol),
fatty acids containing from 12 to 26 carbon atoms, for instance oleic acid;
dialkyl carbonates, the two alkyl chains possibly being identical or different, such as dicaprylyl carbonate sold under the name Cetiol CC® by Cognis; and
non-volatile oils of high molecular mass, for example between 400 and 10 000 g/mol, in particular between 650 and 10 000 g/mol, for instance:

i) vinylpyrrolidone copolymers such as the vinylpyrrolidone/1-hexadecene copolymer, Antaron® V-216 sold or manufactured by the company ISP (MW=7300 g/mol);

ii) esters such as:

a) linear fatty acid esters with a total carbon number ranging from 35 to 70, for instance pentaerythrityl tetrapelargonate (MW=697.05 g/mol);

b) hydroxylated esters such as polyglycerol-2 triisostearate (MW=965.58 g/mol);

c) aromatic esters such as tridecyl trimellitate (MW=757.19 g/mol), C₁₂-C₁₅ alcohol benzoate, the 2-phenylethyl ester of benzoic acid, and butyloctyl salicylate;

d) esters of C₂₄-C₂₈ branched fatty acids or fatty alcohols such as those described in patent application EP 0 955 039, and especially triisoarachidyl citrate (MW=1033.76 g/mol), pentaerythrityl tetraisononanoate (MW=697.05 g/mol), glyceryl triisostearate (MW=891.51 g/mol), glyceryl tris(2-decyl)tetradecanoate (MW=1143.98 g/mol), pentaerythrityl tetraisostearate (MW=1202.02 g/mol), polyglyceryl-2 tetraisostearate (MW=1232.04 g/mol) or else pentaerythrityl tetrakis(2-decyl)tetradecanoate (MW=1538.66 g/mol);

e) esters and polyesters of dimer diol and of monocarboxylic or dicarboxylic acid, such as esters of dimer diols and of fatty acid and esters of dimer diols and of dimer dicarboxylic acid, such as Lusplan DD-DA5® and Lusplan DD-DA7® sold by the company Nippon Fine Chemical and described in patent application US 2004/175 338, the content of which is incorporated into the present application by reference;
and mixtures thereof.

It may also be mention of fatty alcohols.

As examples of fatty alcohols that may be used according to the invention, mention may be made of linear or branched fatty alcohols, of synthetic origin or alternatively of natural origin, for instance alcohols derived from plant material (coconut, palm kernel, palm, etc.) or animal material (tallow, etc.), other long-chain alcohols may also be used, for instance ether alcohols or Guerbet alcohols. As particular examples of fatty alcohols that may be used in the context of the present invention, mention may be made especially of lauryl alcohol, myristyl alcohol, cetyl alcohol, stearyl alcohol, isostearyl alcohol, palmityl alcohol, oleyl alcohol, cetearyl alcohol (mixture of cetyl alcohol and stearyl alcohol), behenyl alcohol, erucyl alcohol, arachidyl alcohol, 2-hexyldecyl alcohol, isocetyl alcohol and octyldodecanol, and mixtures thereof. Preferably, the fatty alcohol is octyldodecanol.

An emollient active agent according to the invention may be formed by a single oil or a mixture of oils. Advantageously, this or these oil(s) may be used in their native form, i.e. in a form not formulated with an associated compound.

As mentioned previously, it may be advantageous to use this or these oils with heating.

This heating may be performed at a temperature ranging from 50° C. to 250° C., in particular greater than 150° C., or even greater than 180° C.

Preferably, the emollient active agent according to the invention is a non-volatile or sparingly volatile oil in particular chosen from oils of mineral origin such as petrolatum, hydrocarbon-based polar oils such as triglycerides consisting of fatty acid esters of glycerol, especially the avocado oil, and fatty alcohols, in particular Guerbet alcohols, especially octyldodecanol, and mixtures thereof.

More preferably, the emollient active agent according to the invention is a non-volatile or sparingly volatile oil chosen from petrolatum, the avocado oil, octyldodecanol, and mixtures thereof.

### c) Waxes

According to one embodiment variant, the emollient active agent according to the invention may be a wax.

The waxes under consideration in the context of the present invention are chosen from waxes that are solid at room temperature, of animal, plant, mineral or synthetic origin, and mixtures thereof.

As illustrations of waxes that are suitable for the invention, mention may be made especially of hydrocarbon-based waxes, for instance beeswax, lanolin wax, Chinese insect waxes, rice bran wax, carnauba wax, candelilla wax, ouricury wax, esparto grass wax, berry wax, shellac wax, Japan wax and sumach wax; montan wax, orange wax and lemon wax, microcrystalline waxes, paraffins and ozokerite; polyethylene waxes, the waxes obtained by Fischer-Tropsch synthesis and waxy copolymers, and also esters thereof.

Mention may also be made of waxes obtained by catalytic hydrogenation of animal or plant oils containing linear or branched C₈-C₃₂ fatty chains. Among these waxes that may especially be mentioned are isomerized jojoba oil such as the trans-isomerized partially hydrogenated jojoba oil manufactured or sold by the company Desert Whale under the commercial reference Iso-Jojoba-50®, hydrogenated sunflower oil, hydrogenated castor oil, hydrogenated coconut oil, hydrogenated lanolin oil and bis(1,1,1-trimethylolpropane) tetrastearate sold under the name Hest 2T-4S® by the company Heterene.

Mention may also be made of silicone waxes (C₃₀₋₄₅ alkyl dimethicone) and fluoro waxes.

The waxes obtained by hydrogenation of castor oil esterified with cetyl alcohol, sold under the names Phytowax ricin 16L64® and 22L73® by the company Sophim, may also be used. Such waxes are described in patent application FR-A-2 792 190.

A wax that may be used is a C₂₀-C₄₀ alkyl (hydroxystearyloxy)stearate (the alkyl group containing from 20 to 40 carbon atoms), alone or as a mixture.

Such a wax is especially sold under the names Kester Wax K 82 P®, Hydroxypolyester K 82 P® and Kester Wax K 80 P® by the company Koster Keunen.

As microwaxes that may be used, mention may be made especially of carnauba microwaxes such as the product sold under the name MicroCare 350® by the company Micro Powders, synthetic microwaxes such as the product sold under the name MicroEase 114S® by the company Micro Powders, microwaxes formed from a mixture of carnauba wax and polyethylene wax, such as those sold under the names MicroCare 300® and 310® by the company Micro Powders, microwaxes formed from a mixture of carnauba wax and synthetic wax, such as the product sold under the name MicroCare 325® by the company Micro Powders, polyethylene microwaxes such as those sold under the names Micropoly 200®, 220®, 220L® and 250S® by the company Micro Powders, and polytetrafluoroethylene microwaxes such as those sold under the names Microslip 519® and 519 L® by the company Micro Powders.

### d) Thiol Derivative

According to one embodiment variant, the emollient active agent according to the invention may be a thiol derivative.

Among the thiol derivatives that are suitable for use in the invention, mention may be made in particular of:

thioglycolic acid, thiolactic acid, glyceryl monothioglycolate, cysteamine, N-acetylcysteamine, N-propionylcysteamine, cysteine, N-acetylcysteine, thiomalic acid, pantetheine, 2,3-dimercaptosuccinic acid, N-(mercaptoalkyl)-ω-hydroxyalkylamides, N-mono- or N,N-dialkylmercapto-4-butyramides, aminomercaptoalkylamides, derivatives of N-(mercaptoalkyl)succinamic acids and of N-(mercaptoalkyl)succinimides, alkylamino-mercaptoalkylamides, the azeotropic mixture of 2-hydroxypropyl thioglyconate and (2-hydroxy-1-methyl)ethyl thioglycolate, mercaptoalkylaminoamides, N-mercaptoalkylalkanediamides and derivatives of formamidine sulfinic acid, and salts thereof.

According to a preferred embodiment, an emollient active agent according to the invention is featured by at least one thiol derivative, chosen in particular from cysteine and thioglycolic acid.

According to another preferred embodiment, an emollient active agent according to the invention is featured by at least one thiol derivative, chosen in particular from cysteine and thioglycolic acid, combined with at least one ionic liquid, in particular guanidinium carbonate.

Advantageously, the emollient active agent is at least cysteine and it is preferably the cysteine/guanidine carbonate couple. This embodiment is most particularly advantageous with regard to the nails.

More particularly, when the thiol derivative is combined with a guanidinium compound, the working pH may be adjusted and especially to a value below 10.

When the thiol derivative is not combined with a guanidinium compound, its working pH is generally above 10 and better still above 11. However, it may be adjusted to a value below 10 if it is combined with an input of temperature of at least 40° C. for a contact time of 10 minutes, or of 140° C. for a contact time of 2 seconds.

In general, the thiol derivative or the mixture of thiol derivatives is used in a proportion of from 1% to 20% by weight and in particular from 2% to 10% by weight of a composition containing it.

### e) Phosphine

According to one embodiment variant, the emollient active agent may be at least one phosphine.

As non-limiting illustrations of phosphines that are suitable for use in the invention, mention may be made especially of tris(hydroxymethyl)phosphine, tris(hydroxypropyl)phosphine, tris(carboxyethyl)phosphine and tris(propionyl)phosphine.

Preferably, the emollient active agent is of phosphonic nature and is at least tris(propionyl)phosphine.

In general, the phosphine or the mixture of phosphines is used in a proportion of from 1% to 20% by weight and in particular from 2% to 10% by weight of a composition containing it.

### f) Acids or Alkaline Bases

According to one embodiment variant, the emollient active agent may be at least one acid or one alkaline base.

As non-limiting illustrations of the acids and alkaline bases that are suitable for use in the invention, mention may be made especially of salicylic acid, glycolic acid, citric acid, lactic acid, sodium hydroxide, potassium hydroxide, ammonia, monoethanolamine, triethanolamine, calcium hydroxide, alkaline salts of amino acids, urea and urea derivatives, and guanidine derivatives, in particular guanidine hydroxide and guanidine carbonate.

Alkaline salts of amino acids that may be mentioned more particularly include glycinate salts.

Preferably, the emollient active agent is an acid, and especially salicylic acid.

Preferably, the emollient active agent is a base chosen from: potassium hydroxide, ammonia, monoethanolamine, triethanolamine, calcium hydroxide, alkaline salts of amino acids, in particular glycinate salts, urea and urea derivatives, guanidine derivatives, and mixtures thereof.

Preferably, the guanidine derivative is chosen from guanidine hydroxide and guanidine carbonate and is more particularly guanidine carbonate. In general, the acid(s) or alkaline base(s) are used in a proportion of from 0.1% to 20% by weight and in particular from 1% to 10% by weight of a composition containing it.

Among all the abovementioned emollient active agents, the ones that prove to be particularly advantageous are emollient active agents such as:
cysteine and derivatives thereof, such as acetylcysteine,
cysteamine and derivatives thereof, and especially cysteamine amides such as N-acetylcysteamine,
thioglycolic, thiolactic or mercaptopropionic acids and derivatives thereof, especially the glycerol ester of thioglycolic acid,
oils such as polar or non-polar carbon-based or silicone oils,
waxes,
ionic liquids comprising a cationic aromatic compound, for example those based on alkylimidazolium or alkylpyridinium, and
bases chosen from potassium hydroxide, ammonia, monoethanolamine, triethanolamine, calcium hydroxide, alkaline salts of amino acids, in particular glycinate salts, urea and urea derivatives, guanidine derivatives, in particular guanidine hydroxide and guanidine carbonate, and more particularly guanidine carbonate, and mixtures thereof.

According to a particular mode, the emollient active agent used is a mixture of cysteine or derivative and of a base chosen from potassium hydroxide, ammonia, monoethanolamine, triethanolamine, calcium hydroxide, alkaline salts of amino acids, in particular glycinate salts, urea and urea derivatives, guanidine derivatives, in particular guanidine hydroxide and guanidine carbonate, and more particularly guanidine carbonate.

Cosmetic Active Agent

As emerges from the foregoing text, the cosmetic active agent for the purposes of the present invention is an active agent that is intended to be incorporated into the keratin material under consideration and more particularly into keratin at the surface of the said keratin material.

The process according to the invention is more precisely directed towards promoting the incorporation of this active agent in significant amount, at the surface of the keratin material.

As emerges from the examples below, the treatment with the abovementioned emollient active agent is precisely advantageous for gaining access to such a degree of incorporation.

Thus, the cosmetic active agent is incorporated in a proportion of at least 1% and preferably at least 4% by weight of solid or hydrophobic material into the volume formed by the first 10 micrometres.

As mentioned above, the cosmetic active agent may or may not be a solid material.

In terms of chemical nature, the cosmetic active agent may be mineral or organic.

When this active agent is organic, it may also be of monomeric, oligomeric or polymeric nature.

Specifically, certain materials are most particularly advantageous for reinforcing the mechanical properties of keratin materials. They are more particularly defined in the chapter below on the hardener or reinforcer.

Other materials prove for their part to be advantageous for affording valuable surface properties to keratin materials, such as a hydrophobic or oleofugal property. Such materials, also referred to as “non-hardening materials”, are described below.

A solid active agent is generally advantageous for affording reinforcement of the mechanical properties of keratin.

However, solid active agents may also be advantageous for affording a particular optical effect on a keratin material. Representatives of such active agents that may especially be mentioned include coloured mineral materials, such as pigments. These pigments are also more particularly described below.

### a) Hardener or Reinforcer

According to one aspect of the invention, the cosmetic active agent under consideration is a hardener or reinforcer.

As mentioned above, an active agent is termed a “hardener” or “reinforcer” with regard to its capacity for reinforcing the mechanical properties of a keratin material.

After the process according to the invention, the hardener or reinforcer is generally incorporated into the keratin, contiguous to the outer surface of the keratin material under consideration, and more particularly into a thickness of keratin ranging from 0.1 to 40 micrometres.

According to a first variant, this active agent is incorporated as such into the keratin at the surface of the said keratin material, like, for example, generally uncoloured organic or mineral solid particles especially such as silica or alkoxysilane derivatives as described below.

According to a second variant, this active agent is capable of forming in situ in the keratin a solid material like, for example, a siccative oil.

According to a third variant, this active agent is capable of generating in situ a composite by interacting or not interacting with the keratin incorporating it. In this last embodiment, it may especially be a crosslinking agent that is capable of interacting with reactive functions of keratin, a polymer, a monomer or oligomer capable of polymerizing in situ, in keratin, or may be featured by a mixture of two compounds that are capable of condensing in situ in keratin.

Alkoxysilane Compound

In the context of the present invention, the term “alkoxysilane” means a compound comprising at least one silicon atom bearing at least one alkoxy group, preferably two, three or four alkoxy groups.

According to a first embodiment, the alkoxysilane compound is of formula R¹ₓSi(OR²)₍₄₋ₓ₎ (I) and may be chosen from the compounds of formulae (Ia) and (Ib) below:

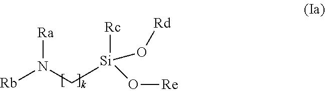

in which:
Ra and Rb represent, independently, a hydrogen atom or a (C₁-C₂₀)alkyl group, such as a methyl group or a cyclohexyl group, or an aryl group, such as a phenyl or a benzyl, or a (C₁-C₂₀)aminoalkyl group, or a (C₁-C₂₀)hydroxyalkyl group, or a (C₁-C₁₀)alkoxy group, or a group of formula (III) or (IV) below:

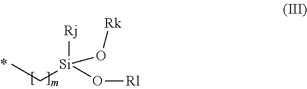

with Rj representing a (C₁-C₂₀)alkyl group such as a methyl or a (C₁-C₁₀)alkoxy group, preferably an ethoxy group,
with Rk and Rl, independently, representing a (C₁-C₁₀)alkyl group, preferably an ethyl group,

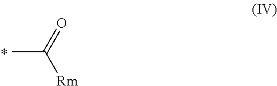

with Rm representing a (C₁-C₂₀)alkyl group or a (C₁-C₁₀)alkoxy group, such as a methoxy, or an amino group,
Ra and Rb possibly being linked to form a ring, for example a cyclohexyl group,
Rc represents, independently, a (C₁-C₂₀)alkyl group such as a methyl or a (C₁-C₁₀)alkoxy group, preferably an ethoxy group,
Rd and Re, independently, represent a (C₁-C₁₀)alkyl group, preferably an ethyl group,
Rf represents a hydrogen atom or a (C₁-C₂₀)alkyl group, or a group of formula (V) below:

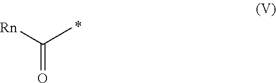

with Rn representing a (C₁-C₂₀)alkyl group, preferably a methyl group,
k and m represent, independently, an integer between 1 and 20, preferably between 1 and 3, preferably equal to 1 or 3.

Among the alkoxysilane compounds of formula (Ia), mention may be made especially of 3-aminopropyltriethoxysilane (APTES),3-aminopropylmethyldiethoxysilane (APMDES) and the oligomers formed from APTES or from APMDES or else N-cyclohexylaminomethyltriethoxysilane.

APTES may, for example, be sold by the company Dow Corning under the name Xiameter OFS-6011 Silane or under the name APTES Silsoft A-1100 by the company Momentive Performance Materials.

APMDES may be sold, for example, by the company Evonik under the name Dynasylan 1505.

The N-cyclohexylaminomethyltriethoxysilane may be sold, for example, by the company Wacker under the name Geniosil XL 926.

Preferably, the alkoxysilane compound is 3-aminopropyltriethoxysilane (APTES).

According to another embodiment, the alkoxysilane may be chosen from the compounds of formulae Si(OR^{2′})₄; R³_{x′}Si(OR^{2′})_{(4-x′)} and [R³_{y′}(OR^{2′})_{z′}SiO_{((4-y′-z′)/2)}]_{n′}, in which:

R³ represents, independently:
a (C₁-C₂₀)alkyl group, which can be optionally substituted with a (meth)acrylate group, an acetoxy group or a glycidoxy group,
an aryl group, such as a phenyl or benzyl group, or
a fluoroalkyl group such as the tridecafluorooctyl group,

R^{2′} represents, independently, a hydrogen atom or a (C₁-C₁₀)alkyl group,
x′ represents 1 or 2,
y′ represents, independently, 0, 1 or 2,
z′ represents, independently, 0, 1, 2 or 3,

the sum of y′ and z′ being less than or equal to 3,
n′ represents an integer between 2 and 1000.

Among the alkoxysilane compounds of formulae Si(OR^{2′})₄; R³_{x′}Si(OR^{2′})_{(4-x′)} and [R³_{y′}(OR^{2′})_{z′}SiO_{((4-y′-z′)/2)}]_{n′}, mention may be made of tetraethoxysilane (TEOS), methyltriethoxysilane (MTES), octyltriethoxysilane (OTES), dimethyldiethoxysilane (DMDES), diethyldiethoxysilane, dipropyldiethoxysilane, propyltriethoxysilane, isobutyltriethoxysilane, phenyltriethoxysilane, phenylmethyldiethoxysilane, diphenyldiethoxysilane, benzyltriethoxysilane, benzylmethyldiethoxysilane, dibenzyldiethoxysilane and acetoxymethyltriethoxysilane, and mixtures thereof.

The MTES may be sold, for example, by the company Evonik under the name Dynasylan.

Preferably, the alkoxysilane compound of formulae Si(OR^{2′})₄; R³_{x′}Si(OR^{2′})_{(4-x′)} and [R³_{y′}(OR^{2′})_{z′}SiO_{((4-y′-z′)/2)}]_{n′}, is chosen from methyltriethoxysilane (MTES), propyltriethoxysilane (PTES) and octyltriethoxysilane (OTES).

According to one embodiment, the alkoxysilanes may be used in a prehydrolysed form in order to accelerate their reaction and their stability over time. To do this, the alkoxysilane is placed in contact with a molar equivalent (from 1/4 to 4/1), preferably under acidic conditions and until hydrolysis of the alkoxysilane is obtained. The evolution of this hydrolysis reaction may be monitored by measuring the amount of ethanol liberated.

Preferably, the hardener is at least one alkoxysilane compound, in particular (3-aminopropyl)triethoxysilane (APTES), methyltriethoxysilane (MTES) or octyltriethoxysilane (OTES).

Preferably, the alkoxysilane compound used according to the invention is methyltriethoxysilane (MTES).

Organic or Mineral Solid Particles

These may be colourless or white, mineral or synthetic particles of any form.

More particularly, the particles may be chosen from mineral compounds, such as silica, titanium, or transition metals, or organic compounds. Examples that may be mentioned include:
silica microspheres, especially of open porosity or, preferably, hollow silica microspheres, such as the products Silica Beads SB 700/HA or Silica Beads SB 700 from the company Maprecos;
microporous polymer microspheres, which have a structure similar to that of a sponge; they generally have a specific surface area of at least 0.5 m²/g and in particular of at least 1 m²/g, said specific surface area having no upper limit other than that resulting from the practical possibility of making microspheres of very high porosity: the specific surface area may, for example, be up to 1000 m²/g or even more. Microspheres that may be mentioned include acrylic polymer microspheres, such as those made of crosslinked acrylate copolymer Polytrap 6603 Adsorber from the company RP Scherer, and those made of polymethyl methacrylate Micropearl M 100 from the company SEPPIC;
polyurethane powder, such as the powdered copolymer of hexamethylene diisocyanate and of trimethylol hexyl lactone sold under the names Plastic Powder D-400 and T-7 by the company Toshiki;
polymer microcapsules that comprise a single closed cavity and form a reservoir, which may contain a liquid, especially a cosmetic active agent; they are prepared via known processes such as those described in U.S. Pat. No. 3,615,972 and EP-A-0 56219. They may be made, for example, of polymers or copolymers of ethylenically unsaturated acid, amine or ester monomers, of urea-formaldehyde polymers or of vinylidene chloride polymers or copolymers; by way of example, mention may be made of microcapsules made of methyl acrylate or methacrylate polymers or copolymers, or alternatively of copolymers of vinylidene chloride and of acrylonitrile; among these polymers, mention will be made especially of those containing 20-60% by weight of units derived from vinylidene chloride, 20-60% by weight of units derived from acrylonitrile and 0-40% by weight of other units such as units derived from an acrylic and/or styrene monomer; crosslinked acrylic polymers or copolymers may also be used;
elastomeric crosslinked organopolysiloxane spherical powders, described especially in document JP-A-02 243 612, such as those sold under the name Trefil Powder E-506C by the company Dow Corning;
the carnauba wax microbeads sold under the name Microcare 350® by the company Micro Powders and the paraffin wax microbeads sold under the name Microease 114S® by the company Micro Powders;
metal soaps in powder form. Among these soaps, mention may be made especially of metal soaps of fatty acids containing from 12 to 22 carbon atoms and in particular those containing from 12 to 18 carbon atoms. The metal of the metal soap may especially be zinc or magnesium. The fatty acid may be chosen especially from lauric acid, myristic acid, stearic acid and palmitic acid. The metal soaps that may be used include zinc laurate, magnesium stearate, magnesium myristate and zinc stearate, and mixtures thereof;
talcs or hydrated magnesium silicates, especially in the form of particles generally less than 40 μm in size;
micas or aluminosilicates of varied compositions that are especially in the form of flakes from 2 to 200 μm and preferably from 5 to 70 μm in size, and from 0.1 to 5 μm and preferably 0.2 to 3 μm in thickness, these micas possibly being of natural origin (for example muscovite, margarite, roscoelite, lepidolite or biotite) or of synthetic origin;
clays such as sericites, which belong to the same chemical and crystalline class as muscovite;
kaolin or hydrated aluminium silicate, which is especially in the form of particles of isotropic forms generally less than 30 μm in size;
boron nitrides;
powders of tetrafluoroethylene polymers, such as Ceridust 9205 F from the company Clariant;
precipitated calcium carbonate, especially in the form of particles greater than 10 μm in size;
magnesium carbonate and magnesium hydrogen carbonate;
hydroxyapatite;
powders of non-expanded synthetic polymers, such as polyethylene, polyesters (for example polyethylene isophthalate or terephthalate) and polyamides (for example Nylon), in the form of particles less than 50 μm in size;
powders of spheronized, crosslinked or non-crosslinked synthetic polymers, for instance polyamide powders such as poly-β-alanine powder or Nylon powder, for example Orgasol powder from the company Atochem, polyacrylic acid or polymethacrylic acid powder, powders of polystyrene crosslinked with divinylbenzene, and silicone resin powders;
bismuth oxychloride powders;
powders of organic materials of natural origin, for instance starches, especially corn starch, wheat starch or rice starch;
and mixtures thereof.

Siccative Oil

A hardener according to the invention may also be a siccative oil.

The term “siccative oil” is intended to denote an oil which, when spread as a thin coat and then exposed to the air, transforms into a solid film.

In particular, in the context of the present invention, the term “siccative oil” is intended to denote oils, and preferably triglycerides, comprising conjugated double bonds, preferably comprising at least two conjugated double bonds and preferably comprising at least three conjugated double bonds.

The siccative oils in accordance with the invention may be of natural origin.

Advantageously, the siccative oil may be chosen from siccative plant oils such as linseed oil, Chinese (or Cantonese) wood oil, oiticica oil, vernonia oil, poppy oil, pomegranate oil or marigold oil; esters of these plant oils, or alkyd resins obtained from these plant oils, and mixtures thereof.

Alkyd resins are polyesters comprising hydrocarbon-based fatty acid chains, obtained especially by polymerization of polyols and polyacids or of the corresponding anhydride thereof, in the presence of fatty acids. These fatty acids are present especially in the form of triglycerides, which are in the majority natural oils, in particular such as the oils mentioned previously.

The siccative oil that is suitable for use in the present invention may be modified by chemical reaction.

In particular, it may be refined and/or partially polymerized. In this respect, mention may be made of blown oils and stand oils, and maleinized, epoxidized or boiled oils.

The blowing of an oil is characterized especially by polymerization of the said oil with atmospheric oxygen.

According to one particular embodiment of the invention, the siccative oil is a refined, modified or blown linseed oil.

Crosslinking Agent

As mentioned above, such an agent is capable of generating in situ in keratin a composite.

Among the crosslinking agents that may be used, mention may be made of any compound that is capable of generating a three-dimensional network. It therefore generally comprises at least two functional sites capable of reacting simultaneously or in sequence to establish bonds with the keratin molecules and/or other molecules of the crosslinking agent to create the crosslinking effect, a molecule may react with two functions of keratin. For example, in other variants, several crosslinking molecules will react with keratin and will react together to create the expected three-dimensional network.

As non-limiting illustrations of these crosslinking agents, mention may be made especially of multifunctional monomers of diacrylate, dimethacrylate, dialdehyde, diepoxide or dianhydride type. Such monomers are capable of reacting with the nucleophilic functions of keratin (hydroxyl, amines, thiols), and are optionally capable of reacting with each other.

More precisely, mention may be made of allyl(meth)acrylate, cinnamyl(meth)acrylate, glycidyl(meth)acrylate, pentaerythrityl tetra(meth)acrylate, ethylene glycol di(meth)acrylate, pentaerythrityl tri(meth)acrylate, divinylbenzene, trivinylbenzene, diallyl phthalate, (poly)ethylene glycol dimethacrylate, methylenebisacrylamide, 1,3-butanediol di(meth)acrylate, 1,4-butanediol di(meth)acrylate, 1,6-hexanediol di(meth)acrylate, neopentyl glycol di(meth)acrylate, bisphenol-A di(meth)acrylate, trimethylolpropane tri(meth)acrylate, monomers comprising an isocyanate side function, which, after addition of diamine, dialcohol or amino alcohol, will form a urea or urethane bond, diallyl ether, triallyl cyanurate, diallyl maleate, dipropylene glycol diallyl ether, polyglycol diallyl ethers, triethylene glycol divinyl ether, hydroquinone diallyl ether, tetraallyloxyethane, tetra- or diethylene glycol di(meth)acrylate, triallylamine, tetraallylethylenediamine, trimethylolpropane diallyl ether, trimethylolpropane triacrylate, methylenebis(meth)acrylamide or divinylbenzene, allylic ethers of alcohols of the sugar series, or other allyl or vinyl ethers of polyfunctional alcohols, and also allylic esters of phosphoric and/or vinylphosphonic acid derivatives, or mixtures of these compounds.

More particularly, mention may be made of formaldehyde and mono- or multifunctional aldehydes.

Polymer

According to another embodiment variant, the hardener may be a polymer.

For the purposes of the present invention, the polymers are more particularly carbon-based or silicone film-forming polymers.

Film Forming Polymer

For the purposes of the present invention, the term “film-forming polymer” denotes a polymer that is capable of forming an isolable and in particular continuous and adherent film, on a keratin support.

This film-forming polymer may be chosen from the group formed by synthetic polymers of radical type or of polycondensate type, and polymers of natural origin, and mixtures thereof.

A film-forming polymer that is suitable for use in the invention may be chosen in particular from:
polysaccharides. Among the polysaccharides that are suitable for use in the invention, examples that may be mentioned include cellulose esters and ethers, such as nitrocellulose, cellulose acetate, cellulose acetobutyrate, cellulose acetopropionate and ethylcellulose, or alternatively optionally modified guar gum, such as ethylguar;
synthetic polymers such as polyurethanes, acrylic polymers, vinyl polymers, polyvinyl butyrals, alkyd resins and ketone/aldehyde resins, resins derived from aldehyde condensation products, such as arylsulfonamide-formaldehyde resins, for instance toluenesulfonamide-formaldehyde resin, arylsulfonamide-epoxy resins or ethyl tosylamide resins;
polymers of natural origin, such as plant resins, such as dammar resins, elemi gums, copal resins, and benzoin; gums such as shellac, sandarac gum and gum mastic.

Use may in particular be made, as film-forming polymers, of the toluenesulfonamide/formaldehyde resins Ketjentflex MS80 from Akzo or Santolite MHP or Santolite MS 80 from Faconnier or Resimpol 80 from Pan Americana, the alkyd resin Beckosol ODE 230-70-E from Dainippon, the acrylic resin Acryloid B66 from Rohm & Haas, the polyurethane resin Trixene PR 4127 from Baxenden or the acetophenone/formaldehyde resin sold under the reference Synthetic Resin SK by Degussa.

According to one particular embodiment, the film-forming polymer is chosen from polysaccharides or polysaccharide derivatives, preferably from cellulose ethers and esters.

The film-forming polymer according to the invention is preferably used with a solvent or a vehicle.

Wax

Among the waxes under consideration in the context of the present invention, mention may be made of those described previously. Among the waxes, mention may be made in particular of ceramides.

According to another embodiment variant, the hardener may be a ceramide compound.

The term “ceramide” in particular denotes compounds corresponding to the following formula:

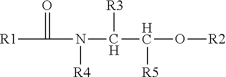

in which

R₁ denotes:
either a linear or branched, saturated or unsaturated C₁-C₅₀ and preferably C₅-C₅₀, hydrocarbon-based radical, this radical possibly being substituted with one or more and preferably from one to six hydroxyl groups optionally esterified with an acid R₇COOH, R₇ being a saturated or unsaturated, linear or branched, optionally mono- or polyhydroxylated C₁-C₃₅ hydrocarbon-based radical preferably with from one six hydroxyl groups, the hydroxyls of the radical R₇ possibly being esterified with a saturated or unsaturated, linear or branched C₁-C₃₅ fatty acid, optionally mono- or polyhydroxylated preferably with from one six hydroxyl groups,
or a radical R″—(NR—CO)—R′, R denotes a hydrogen atom or a linear or branched, saturated or unsaturated C₁-C₂₀ hydrocarbon-based radical which is mono- or polyhydroxylated preferably with from one to six hydroxyl groups (preferentially monohydroxylated), R′ and R″ are linear or branched, saturated or unsaturated hydrocarbon-based radicals, in which the sum of the carbon atoms is between 9 and 30, R′ being a divalent radical,
or a radical R₈—O—CO—(CH₂)p, R₈ denoting a linear or branched, saturated or unsaturated C₁-C₂₀ hydrocarbon-based radical, p being an integer ranging from 1 to 12;

R₂ is chosen from a hydrogen atom, a radical of saccharide type, in particular a (glycosyl)ₙ, (galactosyl)ₘ or sulfogalactosyl radical, a sulfate or phosphate residue, a phosphorylethylamine radical and a phosphorylethylammonium radical, in which n is an integer ranging from 1 to 4 and m is an integer ranging from 1 to 8;

R₃ denotes a hydrogen atom or a linear or branched, saturated or unsaturated C₁-C₃₃ hydrocarbon-based radical, hydroxylated preferably with from one to six hydroxyl groups or non-hydroxylated, the hydroxyls possibly being esterified with a mineral acid or an acid R₇—COOH, R₇ having the same meanings as above, the hydroxyls possibly being etherified with a (glycosyl)ₙ, (galactosyl)ₘ, sulfogalactosyl, phosphorylethylamine or phosphorylethylammonium radical, n is an integer ranging from 1 to 4 and m is an integer ranging from 1 to 8, R₃ also possibly being substituted with one or more C₁-C₁₄ alkyl radicals;

R₄ denotes a hydrogen atom, a methyl or ethyl radical, a saturated or unsaturated, linear or branched, optionally hydroxylated C₃-C₅₀ hydrocarbon-based radical or a radical —CH₂—CHOH—CH₂—O—R₆ in which R₆ denotes a linear or branched, saturated or unsaturated C₁₀-C₂₆ hydrocarbon-based radical or a radical R₈—O—CO—(CH₂)ₚ, R₈ denotes a linear or branched, saturated or unsaturated C₁-C₂₀ hydrocarbon-based radical, p being an integer ranging from 1 to 12,

R₅ denotes a hydrogen atom or a linear or branched, saturated or unsaturated C₁-C₃₀ hydrocarbon-based radical, optionally mono- or polyhydroxylated preferably with from one to six hydroxyl groups, the hydroxyls possibly being etherified with a (glycosyl)ₙ, (galactosyl)ₘ, with n representing an integer ranging from 1 to 4 and m an integer ranging from 1 to 8, sulfogalactosyl, phosphorylethylamine or phosphorylethylammonium radical,

with the proviso that when R₃ and R₅ denote hydrogen or when R₃ denotes hydrogen and R₅ denotes methyl, then R₄ does not denote a hydrogen atom or a methyl or ethyl radical.

Among the compounds having the above formula, mention may be made most particularly of the ceramides and/or glycoceramides described by Downing in the Journal of Lipid Research, Vol. 35, page 2060, 1994, or those described in French patent application FR-2 673 179.

Among the ceramides that are preferred are those for which R₁ denotes an optionally hydroxylated alkyl or alkenyl radical derived from C₁₄-C₂₂ fatty acids; R₂ denotes a hydrogen atom; and R₃ denotes an optionally hydroxylated saturated linear C₁₁-C₁₇ and preferably C₁₃-C₁₅ radical.

Such compounds are chosen, for example, alone or as a mixture, from:
N-linoleoyldihydrosphingosine,
N-oleoyldihydrosphingosine or 2-oleamido-1,3-octadecanediol,
N-palmitoyldihydrosphingosine,
N-stearoyldihydrosphingosine,
N-behenyldihydrosphingosine,
N-2-hydroxypalmitoyldihydrosphingosine,
N-stearoylphytosphingosine,
N-palmitamidohexadecanediol.

Use may also be made of specific mixtures, for instance mixtures of ceramides 2 and of ceramides 5 according to the Downing classification.

It is also possible to use compounds for which R₁ denotes an alkyl or alkenyl radical derived from C₁₄-C₂₂ fatty acids; R₂ denotes a galactosyl or sulfogalactosyl radical; and R₃ denotes a saturated or unsaturated C₁₄-C₂₂ hydrocarbon-based radical and preferably a —CH═CH—(CH₂)₁₂—CH₃ group.

Compounds of ceramide type are described, for example, in patent applications DE 4424530, DE 4424533, DE 4402929, DE 4420736, WO 95/23807, WO 94/07844, EP 646 572, WO 95/16665, FR 2 673 179, EP 227 994 and WO 94/07844, WO 94/24097 and WO 94/10131, to which reference may be made.

Examples that may be mentioned include the product consisting of a mixture of glycoceramides, sold under the trade name Glycocer® by the company Waitaki International Biosciences.

Use may also be made of the compounds described in patent applications EP 227 994, EP 647 617, EP 736 522 and WO 94/07844.

Such compounds are, for example, Questamide H®, also known as (bis(N-hydroxyethyl-N-cetyl)malonamide) and sold by the company Quest, and cetylic acid N-(2-hydroxyethyl)-N-(3-cetyloxy-2-hydroxypropyl)amide.

Use may also be made of N-docosanoyl-N-methyl-D-glucamine described in international patent application WO 94/24097.

Pasty Fatty Substances

The pasty fatty substance may be chosen from:
polymeric silicone compounds, for instance polydimethylsiloxanes of high molecular masses, polydimethylsiloxanes containing side chains of the alkyl or alkoxy type containing from 8 to 24 carbon atoms, especially stearyl dimethicones,
polymeric fluoro compounds,
vinyl polymers, especially:
olefin homopolymers,
olefin copolymers,
hydrogenated diene homopolymers and copolymers,
linear or branched oligomers, homopolymers or copolymers of alkyl(meth)acrylates preferably containing a C₈-C₃₀ alkyl group,
oligomers, homopolymers and copolymers of vinyl esters containing C₈-C₃₀ alkyl groups,
oligomers, homopolymers and copolymers of vinyl ethers containing C₈-C₃₀ alkyl groups,
liposoluble polyethers resulting from the polyetherification between one or more C₂-C₁₀₀ and preferably C₂-C₅₀ diols,
polyesters,
and mixtures thereof.

Among the liposoluble polyethers, mention may be made especially of copolymers of ethylene oxide and/or of propylene oxide with C₆-C₃₀ alkylene oxides. Preferably, the weight ratio of the ethylene oxide and/or propylene oxide to the alkylene oxides in the copolymer is from 5/95 to 70/30. In this family, mention will be made especially of block copolymers comprising C₆-C₃₀ alkylene oxide blocks with a molecular weight ranging from 1000 to 10 000, for example a polyoxyethylene/polydodecylene glycol block copolymer such as the ethers of dodecanediol (22 mol) and of polyethylene glycol (45 oxyethylene or OE units) sold under the brand name Elfacos ST9 by Akzo Nobel.

Among the polyesters, the following are especially preferred:
non-crosslinked polyesters resulting from the polycondensation between a linear or branched C₄-C₅₀ dicarboxylic acid or polycarboxylic acid and a C₂-C₅₀ diol or polyol; or
polyesters resulting from esterification between a polycarboxylic acid and an aliphatic hydroxylated carboxylic acid ester, such as Risocast DA-L and Risocast DA-H sold by the Japanese company Kokyu Alcohol Kogyo, which are esters resulting from the esterification reaction of hydrogenated castor oil with dilinoleic acid or isostearic acid.

Preferably, the hardener according to the invention is chosen from an alkoxysilane compound, a siccative oil, a ceramide, a polymer and/or a crosslinking agent.

More preferentially, a hardener or reinforcer according to the invention may be chosen from an alkoxysilane compound, such as (3-aminopropyl)triethoxysilane (APTES), methyltriethoxysilane (MTES), octyltriethoxysilane (OTES) or tetraethoxysilane (TEOS), pigmentary particles, a polymer, a monomer or oligomer capable of polymerizing in situ, in keratin, a crosslinking agent such as formaldehyde, mono- or multifunctional aldehydes, and/or a siccative oil.

### b) Non-Hardener

According to another aspect, the cosmetic active agent under consideration according to the invention is a non-hardener.

Preferably, such a non-hardener is chosen from an elastomeric material, a hydrophobic material, an oleofugal material, a diffusing material, a cationic surfactant and/or a cationic polymer.

Non-hardeners according to the invention are particularly advantageous since they improve the mechanical properties of keratin materials, especially the hair. A hardener according to the invention will afford an anti-frizz effect on wet hair, or an anti-greasy, conditioning or beautifying effect on hair ends.

Elastomeric Material

Elastomeric materials that are more particularly under consideration are silicone elastomers, also known as organopolysiloxane elastomers.

The term “organopolysiloxane elastomer” means a supple, deformable organopolysiloxane with viscoelastic properties and especially the consistency of a sponge or a supple sphere. Its modulus of elasticity is such that this material withstands deformation and has a limited ability to extend and to contract. This material is capable of regaining its original shape after stretching.

It is more particularly a crosslinked organopolysiloxane elastomer.

Preferably, the organopolysiloxane elastomer is obtained by crosslinking addition reaction (A) of diorganopolysiloxane containing at least two hydrogens each bonded to a silicon, and (B) of diorganopolysiloxane containing at least two ethylenically unsaturated groups bonded to silicon, especially in the presence (C) of a platinum catalyst, as described, for instance, in patent application EP-A-295 886.

In particular, the organopolysiloxane elastomer may be obtained by reaction of a dimethylpolysiloxane with dimethylvinylsiloxy end groups and of methylhydrogenopolysiloxane with trimethylsiloxy end groups, in the presence of a platinum catalyst.

Compound (A) may especially be chosen from methylhydrogenopolysiloxanes containing trimethylsiloxy end groups, dimethylsiloxane-methylhydrogenosiloxane copolymers containing trimethylsiloxy end groups, dimethylsiloxane-methylhydrogenosiloxane cyclic copolymers.

The organopolysiloxanes (B) may be chosen in particular from methylvinylpolysiloxanes, methylvinylsiloxane-dimethylsiloxane copolymers, dimethylpolysiloxanes containing dimethylvinylsiloxy end groups, dimethylsiloxane-methylphenylsiloxane copolymers containing dimethylvinylsiloxy end groups, dimethylsiloxane-diphenylsiloxane-methylvinylsiloxane copolymers containing dimethylvinylsiloxy end groups, dimethylsiloxane-methylvinylsiloxane copolymers containing trimethylsiloxy end groups, dimethylsiloxane-methylphenylsiloxane-methylvinylsiloxane copolymers containing trimethylsiloxy end groups, methyl(3,3,3-trifluoropropyl)polysiloxanes containing dimethylvinylsiloxy end groups, and dimethylsiloxane-methyl(3,3,3-trifluoropropyl)siloxane copolymers containing dimethylvinylsiloxy end groups.

It is advantageous for the compound (A) to be added in an amount such that the molecular ratio of the total amount of hydrogen atoms bonded to silicon atoms in the compound (A) to the total amount of all the ethylenically unsaturated groups in the compound (B) is within the range from 1.5/1 to 20/1.

The compound (C) is the catalyst of the crosslinking reaction and is in particular chloroplatinic acid, chloroplatinic acid/olefin complexes, chloroplatinic acid/alkenylsiloxane complexes, chloroplatinic acid/diketone complexes, platinum black and platinum-on-support.

The catalyst (C) is preferably added from 0.1 to 1000 parts by weight, better still from 1 to 100 parts by weight, as platinum metal proper, per 1000 parts by weight of the total amount of the compounds (A) and (B).

The organopolysiloxane elastomer particles are generally used conveyed in a form such as, for example, a gel formed from an elastomeric organopolysiloxane included in at least one hydrocarbon-based oil and/or one silicone oil. In these gels, the organopolysiloxane particles are often nonspherical particles.

Non-emulsifying elastomers are especially described in patents EP 242 219, EP 285 886 and EP 765 656 and in patent application JP-A-61-194 009, the content of which is incorporated by way of reference.

Spherical elastomers that may be used include those sold under the names DC 9040, DC 9041, DC 9509, DC 9505 and DC 9506 by the company Dow Corning.

Organopolysiloxane elastomers with groups MQ, such as those sold by the company Wacker under the names Belsil RG100, Belsil RPG33 and, preferentially, RG80 may also be used in the compositions according to the invention. The elastomer may also be an emulsifying elastomer.

The organopolysiloxane elastomer may also be chosen from polyoxyalkylenated organopolysiloxane elastomers.

The polyoxyalkylenated organopolysiloxane elastomer is a crosslinked organopolysiloxane elastomer that may be obtained by crosslinking addition reaction of diorganopolysiloxane containing at least one hydrogen bonded to silicon and of a polyoxyalkylene containing at least two ethylenically unsaturated groups.

Advantageously, the polyoxyalkylenated organopolysiloxane elastomers may be formed from divinyl compounds, in particular polyoxyalkylenes containing at least two vinyl groups, which react with Si—H bonds of a polysiloxane.

Polyoxyalkylenated elastomers are especially described in U.S. Pat. No. 5,236,986, U.S. Pat. No. 5,412,004, U.S. Pat. No. 5,837,793 and U.S. Pat. No. 5,811,487, the content of which is incorporated by reference.

Polyoxyalkylenated organopolysiloxane elastomers that may be used include those sold under the names KSG-21, KSG-20, KSG-30, KSG-31, KSG-32, KSG-33, KSG-210, KSG-310, KSG-320, KSG-330 and KSG-340 by the company Shin-Etsu, and DC9010 and DC9011 by the company Dow Corning.

The organopolysiloxane elastomer may also be chosen from polyglycerolated organopolysiloxane elastomers.

The polyglycerolated organopolysiloxane elastomer according to the invention may be an organopolysiloxane elastomer that may be obtained by crosslinking addition reaction of diorganopolysiloxane containing at least one hydrogen bonded to silicon and of polyglycerolated compounds containing ethylenically unsaturated groups, especially in the presence of a platinum catalyst.

The polyglycerolated organopolysiloxane elastomer according to the invention is conveyed in gel form in at least one hydrocarbon-based oil and/or one silicone oil. In these gels, the polyglycerolated elastomer is often in the form of non-spherical particles.

Polyglycerolated organopolysiloxane elastomers that may be used include those sold under the names KSG-710, KSG-810, KSG-820, KSG-830 and KSG-840 by the company Shin-Etsu.

Non-emulsifying elastomers that may be used more particularly include those sold under the names KSG-6, KSG-15, KSG-16, KSG-18, KSG-41, KSG-42, KSG-43 and KSG-44 by the company Shin-Etsu, DC9040 and DC9041 by the company Dow Corning, and SFE 839 by the company General Electric.

Emulsifying elastomers that may more particularly be used include those sold under the names KSG-31, KSG-32, KSG-33, KSG-210 and KSG-710 by the company Shin-Etsu.

According to another aspect of the invention, the elastomer may also be chosen from non-silicone elastomers.

In particular, the elastomer may be a polyurethane.

Hydrophobic Material

The hydrophobic material may especially be a carbon-based or silicone oil especially as defined above.

More particularly, this hydrophobic material may be an oil bearing reactive functions such as alkene or epoxide functions. Such an oil changes after insertion into keratin. Such is the case for the siccative oils as defined above, and in particular for linseed oil.

Oleofugal Material

Oleofugal materials that are more particularly under consideration are fluoro compounds.

The fluoro compounds may be chosen from perfluoroalkyl phosphates, perfluoropolyethers, polytetrafluoropolyethylene (PTFE), perfluoroalkanes, perfluoroalkylsilazanes, poly(hexafluoropropylene oxides), and polyorganosiloxanes comprising perfluoroalkyl or perfluoropolyether groups.

The term “perfluoroalkyl radical” means an alkyl radical in which all the hydrogen atoms have been replaced with fluorine atoms.

Perfluoropolyethers are especially described in patent application EP 486 135, and sold under the trade name Fomblin by the company Montefluos.

Perfluoroalkyl phosphates are described in particular in patent application JP 0586984. The perfluoroalkyl diethanolamine phosphates sold by Asahi Glass under the reference AsahiGuard AG530 may be used.

Among the linear perfluoroalkanes that may be mentioned are perfluorocycloalkanes, perfluoro(alkylcycloalkanes), perfluoropolycycloalkanes, aromatic perfluoro hydrocarbons (perfluoroarenes) and hydrocarbon-based perfluoro organic compounds comprising at least one heteroatom.

Among the perfluoroalkanes, mention may be made of the linear alkane series such as perfluorooctane, perfluorononane or perfluorodecane.

Among the perfluorocycloalkanes and the perfluoro(alkylcycloalkanes), mention may be made of perfluorodecalin sold under the name Flutec PP5 GMP by the company Rhodia, perfluoro(methyldecalin) and perfluoro(C₃-C₅ alkylcyclohexanes) such as perfluoro(butylcyclohexane).

Among the perfluoropolycycloalkanes, mention may be made of bicyclo[3.3.1]nonane derivatives such as perfluorotrimethylbicyclo[3.3.1]nonane, adamantane derivatives such as perfluorodimethyladamantane, and hydrogenated perfluorophenanthrene derivatives such as tetracosafluorotetradecahydrophenanthrene.

Among the perfluoroarenes, mention may be made of perfluoronaphthalene derivatives, for instance perfluoronaphthalene and perfluoromethyl-1-naphthalene.

Cationic Polymers

According to another aspect, the cosmetic active agent is a non-hardener chosen from cationic polymers.

Generally, for the purposes of the present invention, the term “cationic polymer” denotes any polymer containing cationic groups and/or groups that can be ionized into cationic groups.

The preferred cationic polymers are chosen from those that contain units comprising primary, secondary, tertiary and/or quaternary amine groups that may either form part of the main polymer chain or may be borne by a side substituent directly connected thereto.

Among the cationic polymers, mention may be made more particularly of polymers of the polyamine, polyaminoamide and quaternary polyammonium type and especially those described in French patents FR 2 505 348 and FR 2 542 997.

According to a particular embodiment of the invention, the cationic polymers are chosen from:

(1) homopolymers or copolymers derived from acrylic or methacrylic ester or amide derivatives such as:
copolymers of acrylamide and of dimethylaminoethyl methacrylate quaternized with dimethyl sulfate or with a dimethyl halide, such as the product sold under the name Hercofloc by the company Hercules;
the copolymers of acrylamide and of methacryloyloxyethyltrimethylammonium chloride sold under the name Bina Quat P 100 by the company Ciba Geigy;
the copolymer of acrylamide and of methacryloyloxyethyltrimethylammonium methosulfate sold under the name Reten by the company Hercules;
quaternized or non-quaternized vinylpyrrolidone/dialkylaminoalkyl acrylate or methacrylate copolymers, such as the products sold under the name Gafquat by the company ISP, for instance Gafquat 734 or Gafquat 755, or alternatively the products known as Copolymer 845, 958 and 937. These polymers are described in FR 2 077 143 and FR 2 393 573;
dimethylaminoethyl methacrylate/vinylcaprolactam/vinylpyrrolidone terpolymers, such as the product sold under the name Gaffix VC 713 by the company ISP;
the vinylpyrrolidone/methacrylamidopropyldimethylamine copolymers sold in particular under the name Styleze CC 10 by ISP;
quaternized vinylpyrrolidone/dimethylaminopropylmethacrylamide copolymers such as the product sold under the name Gafquat HS 100 by the company ISP; and
crosslinked polymers of methacryloyloxy(C₁-C₄)alkyltri(C₁-C₄)alkylammonium salts, such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with an olefinically unsaturated compound, in particular methylenebisacrylamide. A crosslinked acrylamide/methacryloyloxyethyltrimethylammonium chloride copolymer (20/80 by weight) in the form of a dispersion containing 50% by weight of the said copolymer in mineral oil may be used more particularly. This dispersion is sold under the name Salcare® SC 92 by the company Ciba. A crosslinked methacryloyloxyethyltrimethylammonium chloride homopolymer containing about 50% by weight of the homopolymer in mineral oil or in a liquid ester may also be used. These dispersions are sold under the names Salcare® SC 95 and Salcare® SC 96 by the company Ciba.

(2) cellulose ether derivatives comprising quaternary ammonium groups, which are described in FR 1 492 597, and in particular the polymers sold under the names “JR” (JR 400, JR 125, JR 30M) or “LR” (LR 400, LR 30M) by the company Union Carbide Corporation. These polymers are also defined in the CTFA dictionary as quaternary ammoniums of hydroxyethylcellulose that have reacted with an epoxide substituted with a trimethylammonium group.

(3) copolymers of cellulose or cellulose derivatives grafted with a water-soluble quaternary ammonium monomer, described especially in U.S. Pat. No. 4,131,576, such as hydroxyalkylcelluloses, for instance hydroxymethyl, hydroxyethyl or hydroxypropyl celluloses grafted especially with a methacryloylethyltrimethylammonium, methacrylamidopropyltrimethylammonium or dimethyldiallylammonium salt.

The products sold corresponding to this definition are, more particularly, the products sold under the name Celquat L 200 and Celquat H 100 by the company National Starch.

(4) non-cellulose cationic polysaccharides described in patents US 2003/589 578 and U.S. Pat. No. 4,031,307, such as guar gums containing trialkylammonium cationic groups. Use is made, for example, of guar gums modified with a 2,3-epoxypropyltrimethylammonium salt (for example, chloride).

Such products are sold especially under the trade names Jaguar C135, Jaguar C15, Jaguar C17 and Jaguar C162 by the company Meyhall.

(5) polymers consisting of piperazinyl units and of divalent alkylene or hydroxyalkylene radicals containing straight or branched chains, optionally interrupted by oxygen, sulfur or nitrogen atoms or by aromatic or heterocyclic rings, and also the oxidation and/or quaternization products of these polymers. Such polymers are described, in particular, in FR 2 162 025 and FR 2 280 361.

(6) water-soluble polyaminoamides prepared in particular by polycondensation of an acidic compound with a polyamine; these polyaminoamides can be crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide or alternatively with an oligomer resulting from the reaction of a difunctional compound which is reactive with a bis-halohydrin, a bis-azetidinium, a bis-haloacyldiamine, a bis-alkyl halide, an epihalohydrin, a diepoxide or a bis-unsaturated derivative; the crosslinking agent being used in proportions ranging from 0.025 to 0.35 mol per amine group of the polyaminoamide; these polyaminoamides can be alkylated or, if they comprise one or more tertiary amine functions, they can be quaternized. Such polymers are described, in particular, in FR 2 252 840 and FR 2 368 508.

Polyaminoamide derivatives resulting from the condensation of polyalkylene polyamines with polycarboxylic acids followed by alkylation with difunctional agents. Mention may be made, for example, of adipic acid/dialkylaminohydroxyalkyldialkylene-triamine polymers in which the alkyl radical is C₁-C₄ and preferably denotes methyl, ethyl or propyl. Such polymers are described in particular in FR 1 583 363.

Among these derivatives, mention may be made more particularly of the adipic acid/dimethylaminohydroxypropyl/diethylenetriamine polymers sold under the name Cartaretine F, F4 or F8 by the company Sandoz.

(7) polymers obtained by reaction of a polyalkylene polyamine containing two primary amine groups and at least one secondary amine group with a dicarboxylic acid chosen from diglycolic acid and saturated C₃-C₈ and preferentially C₃-C₆ aliphatic dicarboxylic acids. The mole ratio of the polyalkylenepolyamine to the dicarboxylic acid being between 0.8:1 and 1.4:1; the polyaminoamide resulting therefrom being reacted with epichlorohydrin in a mole ratio of epichlorohydrin relative to the secondary amine group of the polyaminoamide of between 0.5:1 and 1.8:1. Such polymers are described in particular in U.S. Pat. No. 3,227,615 and U.S. Pat. No. 2,961,347. Polymers of this type are sold in particular under the name Hercosett 57, PD 170 or Delsette 101 by the company Hercules.

(8) alkyldiallylamine or dialkyldiallylammonium cyclopolymers such as homopolymers or copolymers such as the dimethyldiallylammonium chloride homopolymer sold under the name Merquat 100 or Polyquaternium-6 by the company Nalco (and its homologues of low weight-average molecular mass) and the copolymers of diallyldimethylammonium chloride and of acrylamide, sold under the name Merquat 550.

(9) quaternary diammonium polymers such as the tetramethyl hexamethyl hexamethylenediamine/1,3-dichloropropylene polycondensate or the diethyldimethylethylenediamine/1,3-dibromopropylene polycondensate (Polyquaternium-34).

(10) quaternary polyammonium polymers, for instance the products Mirapol A 15, Mirapol AD1, Mirapol AZ1 and Mirapol 175 sold by the company Miranol.

(11) quaternary polymers of vinylpyrrolidone and of vinylimidazole, for instance the products sold under the names Luviquat FC 905, FC 550 and FC 370 by the company BASF.

(12) polylysines, which correspond more particularly to the condensation of several lysine amino acids.

Examples of polylysines that may be mentioned include:
epsilon poly-L-lysine (N=5) sold by Chisso Corporation, which is a 5% solution of polylysine in water;
the product polylysine 10% solution M Grade from Chisso Corporation, which is a 10% solution of polylysine in water;
the product polylysine 25% solution from Chisso Corporation, which is a 25% solution of polylysine in water;
the product polylysine 50% from Chisso Corporation, which is a 50/50 mixture of polylysine (N=25 to 30) with dextrin.

According to one particular embodiment, the polylysine may be a modified polylysine, a polylysine bearing a guanidine or biguanidine function as described in patent application FR 2 851 465, or a thiol-bearing polylysine as described in patent application FR 2 853 533.

The polylysine may be in the form of organic or mineral salts.

The addition salts with an acid are, for example, the hydrochloric, hydrobromic, sulfuric, citric, succinic, tartaric, lactic, para-toluenesulfonic, phosphoric or acetic acid salts, or the salts of fatty acids such as linoleic, oleic, palmitic, stearic, behenic and 18-methyleicosanoic acid.

The addition salts with a base are, for example, the sodium or calcium salts and the salts of hydroxyalkylamines, for instance N-methylglucamine, aminopropanediol or triethanolamine.

(13) polyethyleneimines.

(14) partially hydrolysed polyvinylformamides.

Among the cationic polymers that may be used in the context of the present invention, use is made more advantageously of polymers of families (1), (2), (3), (4), (8) and (9), or mixtures thereof.

Preferably, the cationic polymers used are chosen from families (1), (8) and (9) and even more preferentially those of families (8) and (9). In particular, those corresponding to the dimethyldiallylammonium halide, particularly chloride, homopolymer and/or to the crosslinked homopolymers or copolymers of methacryloyloxy(C₁-C₄)alkyltri(C₁-C₄)alkylammonium salts.

Preferred polymers according to the invention that may particularly be mentioned are Polyquaternium-6, Polyquaternium-34 and the tetramethyl hexamethyl hexamethylenediamine/1,3-dichloropropylene polycondensate.

Preferably, the cationic polymers of the invention are non-silicone and do not contain a fatty chain.

The term “non-silicone cationic polymer not containing a fatty chain” means a polymer which comprises one or more cationic charges, which does not contain any polysiloxane bonds, which preferentially does not comprise any silicon atoms, and which does not contain a fatty chain, i.e. a hydrocarbon-based chain comprising more than 8 carbon atoms and preferentially comprising more than 10 carbon atoms.

Cationic Surfactants

According to another aspect, the cosmetic active agent is a non-hardener chosen from cationic surfactants.

The cationic surfactants may be chosen from:
salts of optionally polyoxyalkylenated primary, secondary or tertiary fatty amines;
quaternary ammonium salts such as tetraalkylammonium, alkylamidoalkyltrialkylammonium, trialkylbenzylammonium, trialkylhydroxyalkylammonium or alkylpyridinium chlorides or bromides, for instance N,N,N-trimethyl-1-docosanaminium chloride (or behentrimonium chloride);
alkylimidazolidiniums such as isostearylethylimidonium ethosulfate,
imidazo line derivatives; and
amine oxides of cationic nature.

Pigments

As mentioned above, a cosmetic active agent may also be featured by a pigment.

The term “pigments” should be understood as meaning white or coloured and inorganic or organic particles which are insoluble in an aqueous solution and which are intended to colour and/or opacify the resulting film.

As inorganic pigments that may be used in the invention, mention may be made of titanium oxide, zirconium oxide or cerium oxide, and also zinc oxide, iron oxide or chromium oxide, ferric blue, manganese violet, ultramarine blue and chromium hydrate.

They may also be pigments having a structure that may be, for example, of sericite/brown iron oxide/titanium dioxide/silica type. Such a pigment is sold, for example, under the reference Coverleaf NS or JS by the company Chemicals and Catalysts, and has a contrast ratio in the region of 30.

The colorant may also comprise a pigment with a structure that may be, for example, of silica microspheres containing iron oxide type. An example of a pigment having this structure is the product sold by the company Miyoshi under the reference PC Ball PC-LL-100 P, this pigment being constituted of silica microspheres containing yellow iron oxide.

Among the organic pigments that may be used in the invention, mention may be made of carbon black, pigments of D&C type, lakes based on cochineal carmine or on barium, strontium, calcium or aluminium, or alternatively the diketopyrrolopyrroles (DPPs) described in documents EP-A-542 669, EP-A-787 730, EP-A-787 731 and WO-A-96/08537.

The term “nacres” should be understood as meaning iridescent or non-iridescent coloured particles of any shape, especially produced by certain molluscs in their shell or alternatively synthesized, which have a colour effect via optical interference.

The nacres may be chosen from nacreous pigments such as titanium mica coated with an iron oxide, mica coated with bismuth oxychloride, titanium mica coated with chromium oxide, titanium mica coated with an organic dye and also nacreous pigments based on bismuth oxychloride. They may also be mica particles at the surface of which are superimposed at least two successive layers of metal oxides and/or of organic dyes.

Examples of nacres that may also be mentioned include natural mica coated with titanium oxide, with iron oxide, with natural pigment or with bismuth oxychloride.

Among the nacres available on the market, mention may be made of the nacres Timica, Flamenco and Duochrome (based on mica) sold by the company Engelhard, the Timiron nacres sold by the company Merck, the Prestige mica-based nacres sold by the company Eckart, and the Sunshine synthetic mica-based nacres sold by the company Sun Chemical.

The nacres may more particularly have a yellow, pink, red, bronze, orange, brown, gold and/or coppery colour or glint.

As illustrations of nacres that may be used in the context of the present invention, mention may be made in particular of gold-coloured nacres sold especially by the company Engelhard under the name Brilliant gold 212G (Timica), Gold 222C (Cloisonne), Sparkle gold (Timica), Gold 4504 (Chromalite) and Monarch gold 233X (Cloisonne); the bronze nacres sold especially by the company Merck under the names Bronze fine (17384) (Colorona) and Bronze (17353) (Colorona) and by the company Engelhard under the name Super bronze (Cloisonne); the orange nacres sold especially by the company Engelhard under the names Orange 363C (Cloisonne) and Orange MCR 101 (Cosmica) and by the company Merck under the names Passion orange (Colorona) and Matte orange (17449) (Microna); the brown-tinted nacres sold especially by the company Engelhard under the names Nu-antique copper 340XB (Cloisonne) and Brown CL4509 (Chromalite); the nacres with a copper glint sold especially by the company Engelhard under the name Copper 340A (Timica); the nacres with a red glint sold especially by the company Merck under the name Sienna fine (17386) (Colorona); the nacres with a yellow glint sold especially by the company Engelhard under the name Yellow (4502) (Chromalite); the red-tinted nacres with a golden glint sold especially by the company Engelhard under the name Sunstone G012 (Gemtone); the pink nacres sold especially by the company Engelhard under the name Tan opale G005 (Gemtone); the black nacres with a golden glint sold especially by the company Engelhard under the name Nu antique bronze 240 AB (Timica); the blue nacres sold especially by the company Merck under the name Matte blue (17433) (Microna); the white nacres with a silvery glint sold especially by the company Merck under the name Xirona Silver; and the golden-green pinkish-orange nacres sold especially by the company Merck under the name Indian summer (Xirona), and mixtures thereof.

Galenical Formulation

For obvious reasons, each of the active agents, namely the emollient active agent and the cosmetic active agent, may be formulated with physiologically acceptable compounds If necessary, to prepare galenical formulations, conventionally proposed for application to the keratin material under consideration.

In general, a galenical formulation containing an associated physiologically acceptable medium is preferred for the application of the cosmetic active agent. On the other hand, in the case of the emollient active agent, it may be used in its pure form such as in the case, for example, of an oil or an ionic liquid.

The choice of such galenical formulations clearly falls within the competence of a person skilled in the art.

The galenical formulations under consideration may be obtained according to the preparation processes conventionally used in cosmetics or dermatology.

Such a composition may especially be a makeup composition, a care composition, a fragrancing composition or a haircare composition, for example as defined below.

Thus, it may be a composition for cleansing or caring for the hair such as a shampoo, a rinse-out or leave-in hair conditioner, a rinse-out composition to be applied before or after dyeing, bleaching, permanent-waving or relaxing the hair or alternatively between the two steps of a permanent-waving or hair-relaxing operation, a hair composition for holding the hairstyle such as a styling lacquer, gel, mousse or spray, or a hair composition such as a hair colouring composition or a composition for permanently reshaping the hair; a makeup composition, and especially a composition for making up the lips, the body, the face or the integuments, such as a foundation, a lipstick, a lip gloss, a face powder, an eyeshadow, a nail varnish, a mascara or an eyeliner; a care composition, and especially a body or facial care composition, or a makeup-removing composition and/or composition for cleansing keratin materials, especially the skin or mucous membranes such as the lips and/or the eyelashes, such as a shower gel, a bath gel or a makeup remover.

The galenical formulations may thus also comprise ingredients conventionally used in the fields concerned.

These ingredients may especially be chosen from surfactants; hair conditioners; opacifiers; fragrances; thickeners; gelling agents; hair dyes; silicone resins; silicone gums; preserving agents; antioxidants; other cosmetic active agents; sunscreens; pH stabilizers; vitamins; moisturizers; antiperspirants; deodorants; self-tanning compounds, and mixtures thereof. The amounts of these various ingredients are those conventionally used in the fields concerned, for example from 0.01% to 20% of the total weight of the composition.

Needless to say, a person skilled in the art will take care to select this or these optional additional compound(s), and/or the amount thereof, such that the advantageous properties of the active agents according to the invention are not, or are not substantially, adversely affected by the addition under consideration.

According to one embodiment, the active agents according to the invention are formulated in a makeup composition. It may be a lipstick and/or a foundation and/or a mascara, and preferably a colourless or coloured nail varnish.

According to another embodiment, the active agent(s) according to the invention are formulated in a care composition, and especially a makeup-removing composition.

It may especially be a composition in the form of an emulsion, for example an O/W, W/O, O/W/O or W/O/W emulsion.

Among the standard adjuvants that may be contained in the aqueous phase and/or in the oily phase of the care compositions in accordance with the invention (according to the water-soluble or liposoluble nature of these adjuvants), mention may be made especially of anionic foaming surfactants such as sodium lauryl ether sulfate, sodium alkyl phosphate, sodium trideceth sulfate; amphoteric foaming surfactants such as alkylbetaines, for instance cocoylbetaine, laurylbetaine and disodium cocoamphodiacetate, and nonionic foaming surfactants such as alkylpolyglucosides (APG); preserving agents; sequestrants (EDTA); antioxidants; fragrances; dyestuffs such as soluble dyes, pigments and nacres; matting, tensioning, whitening or exfoliant fillers; sunscreens; cosmetic or dermatological active agents and hydrophilic or lipophilic agents which have the effect of improving the cosmetic properties of the skin; electrolytes; hydrophilic or lipophilic, anionic, nonionic, cationic or amphoteric, thickening or dispersing polymers. The amounts of these various adjuvants are those conventionally used in the field under consideration, for example from 0.01% to 20% of the total weight of the composition.

As other types of active agent that may be used in the care compositions in accordance with the invention, examples that may be mentioned include water-soluble or liposoluble vitamins, for instance vitamin A (retinol), vitamin E (tocopherol), vitamin C (ascorbic acid), vitamin B5 (panthenol), vitamin B3 (niacinamide), derivatives of these vitamins (especially esters) and mixtures thereof; antiseptics; antibacterial active agents such as 2,4,4′-trichloro-2′-hydroxydiphenyl ether (or triclosan), 3,4,4′-trichlorocarbanilide (or triclocarban); anti-seborrhoeic agents such as salicylic acid; antimicrobial and antibacterial agents such as benzoyl peroxide, salicylic acid, triclosan, azelaic acid, niacin (vitamin PP); enzymes, yeasts, plant extracts such as extracts of tea, mint and water lily, and any other active agent that is suitable for the intended purpose of the composition, and mixtures thereof.

According to another embodiment, the active agents according to the invention are formulated in a hair composition.

The hair compositions according to the invention may also contain cosmetically acceptable adjuvants, for instance surfactants, thickeners, penetrants, fragrances, buffers, and various common adjuvants such as UV-screening agents, waxes, volatile or non-volatile, cyclic or linear or branched, organomodified (especially with amine groups) or non-organomodified silicones, preserving agents, ceramides, pseudoceramides, plant, mineral or synthetic oils, vitamins or provitamins such as panthenol, opacifiers, reducing agents, emulsifiers, preserving agents, fillers, sunscreens, proteins, moisturizers, emollients, softeners, antifoams, antiperspirants, free-radical scavengers, fixing or non-fixing polymers, bactericides, sequestrants, antidandruff agents, antioxidants, basifying agents, and any other additive conventionally used in cosmetic compositions intended to be applied to the hair.

Process According to the Invention

The process according to the invention may be performed at room temperature, with heating and/or under a mechanical stress.

As mentioned above, the placing in contact of the emollient active agent is performed under conditions that are favourable for obtaining the expected softening. Thus, the contact time between the said emollient active agent and the keratin material may vary significantly with regard, firstly, to the chemical nature of the emollient active agent and, secondly, to the type of keratin material.

For example, a more prolonged application time may be required for the nails with regard to their great hardness.

According to one aspect of the invention, the step of applying the emollient active agent and/or the said cosmetic active agent is performed at room temperature.

According to another aspect of the invention, the step of applying the emollient active agent and/or the said cosmetic active agent is performed with heating, more particularly to a temperature of between 50° C. and 250° C. and preferably between 150° C. and 230° C.

The input of heat favourable to the efficacy of the emollient may be conducted using a means specifically intended for heating, for instance a means for propelling hot air such as a hairdryer or a heating device, for example a heating applicator such as a straightening iron when the process is applied to the hair.

For obvious reasons, the adjustment of the input of heat on the keratin material under consideration falls within the competence of a person skilled in the art.

According to another aspect of the invention, the step of applying the emollient active agent and/or the said cosmetic active agent is performed under a mechanical stress, and more particularly it may be performed with a device of roller, brush, comb or pen type or a roll-on applicator.

The invention is illustrated in greater detail by the examples described below, which are given as non-limiting illustrations.

In the examples that follow, the weight percentages are indicated relative to the total weight of the composition. The weight percentages are indicated as weight of starting material.

### EXAMPLES

### Examples 1 to 4

The emollient compositions 1 to 4 below were prepared:

| | Composition | Composition | Composition | Composition |
|---|---|---|---|---|
| | 1 according | 2 according | 3 according | 4 according |
| | to the | to the | to the | to the |
| Compounds | invention | invention | invention | invention |
| L-Cysteine | — | 12 | 12 | — |
| (Ajinomoto) | | | | |
| N-Acetyl | — | — | — | 16.5 |
| L-cysteine | | | | |
| (Wacker | | | | |
| Chimie) | | | | |
| Guanidine | 17.5 | — | 17.5 | 17.5 |
| carbonate | | | | |
| (Palmer | | | | |
| Company) | | | | |
| Citric acid | qs pH 9 | — | — | — |
| Sodium | — | qs pH 9 | — | — |
| hydroxide | | | | |
| Water | qs 100 | qs 100 | qs 100 | qs 100 |
| pH | 9 | 9 | 9 | 9 |

Preparation Process

Compositions 1 to 4 were obtained by mixing the emollient active agents under consideration with water at 20° C.

Evaluation of the Processes

Compositions 1 to 4 are applied to the nails.

At a time t=10 minutes, rinsing is performed.

The hardness of the surface of the nail is evaluated by means of a mechanical rubbed stress performed on 3 mm under a microscope, by placing a steel rod, 1 mm wide and ending in a point, above the surface of the nail. The point of the rod is pressed with a force of 50 g. Next, the nail is slid in order to evaluate the mechanical strength of the surface.

As the surface of the treated nail has become gelled, the Young's modulus of the keratin material of the surface of the nail is considered as being reduced by a factor at least equal to 10, relative to the initial hardness of the nail.

A concentrated aqueous formulation containing 20% by weight of 3-aminopropyltriethoxysilane (referred to as the APTES formulation) (Xiameter OFS-6011 Silane® from the company Dow Corning or APTES Silsoft A-1100® from the company

Momentive Performance Materials) is then applied to the nail.

After drying, further rinsing is performed.

In the case of the nails treated with formulations 1 and 2 and then with the APTES formulation, the nails show hardening, but the effect is much more significant in the case of the nails treated with formulations 3 and 4 according to the invention and then with the APTES formulation.

Conversely, the nails treated with formulations 1 to 4, without treatment with the APTES formulation, are not at all hardened.

The hardness of the surface of the nail is also evaluated by microscopy, by performing the same experiment as described above.

The surface of the nail treated via the process according to the invention is more resistant (no mark left) than the surface of the untreated nail (observation of a mark) and than the surface treated only with one or the other of the two steps.

The same experiment is performed by replacing the APTES formulation with an aqueous formulation containing 20% by weight of methyltriethoxysilane (MTES) (Dynasylan® from the company Evonik), referred to hereinbelow as the prehydrolysed MTES formulation, or with a formulation containing colloidal silica.

The prehydrolysed MTES composition is prepared by introducing 20% by weight of MTES into water. A two-phase composition is obtained.

Next, with stirring, citric acid is added to a pH of about 3.2.

After stirring for 1 hour, the composition becomes monophasic. The reaction brings about a temperature increase.

The formulation containing colloidal silica is a suspension at 30% by weight in water, stabilized at pH 8.9, sold by the company Aldrich under the reference Ludox® AM-30 Colloidal Silica (30 wt % suspension in H₂O).

In the case of the nails treated with formulations 3 and 4 according to the invention, an incrustation effect is obtained.

The surface state of the nail before and after the treatment with formulation 3 is observed by electron microscope with a precision of 500 nm.

It is visually noted that the keratin-softening system modifies the state of the surface of the nail. Its outer surface is softer and thus more able to be penetrated by the additional compound and vertical channels become apparent.

Thus, when the 20% APTES composition is applied at room temperature to the treated surface, then, besides deposition of the APTES at the surface, encrustation of the APTES into the keratin material is observed; the compound has filled the channels, which are no longer apparent. Since the rigidity obtained cannot come solely from the filling of the channels, it is estimated that the softened keratin material and the siliceous material derived from the polymerization of the APTES have become combined.

### Example 5

Bleached hair is treated via various processes:

Process 1:

An 80% solution of 1-ethyl-3-methylimidazolium acetate in water is applied to the hair. To do this, the lock of bleached hair is laid flat in a channel 30 cm long, 1.5 cm wide and 1 cm deep. The composition is then applied to the lock and thus left on for 10 minutes in order to ensure that good impregnation is obtained.

The hair thus treated is then exposed to heating provided by an iron brought to 210° C. The iron is passed over the surface of the treated hair slowly twice and then quickly five times.

Process 2:

An MTES solution as defined in Examples 1 to 4 at 50% in water at pH=3 is applied to the hair, which is then dried.

Process 3:

Hair is successively treated via process 1 and then via process 2.

Process 4:

Avocado oil is applied to the hair. The hair thus coated is then exposed to heating provided by an iron brought to 210° C. The iron is passed over the surface of the hair slowly twice and then quickly five times.

Process 5:

Hair is successively treated via process 4 and then via process 2.

After each of the processes 1 to 5, shampooing is performed twice. The state of the hair is then assessed while wet and then when dry after drying under a styling hood at 40° C.

When the hair is treated via process 1, softening of the hair is observed.

With a treatment according to process 2, the hair is slightly reinforced.

When the hair is treated via process 4, no change in the mechanical state of the hair is noted.

A treatment according to process 3 or according to process 5 affords the hair a styling effect and greater rigidity. These observations are more pronounced when the hair is treated via process 5, the hair being less tacky.

The same test was performed with an APTES formulation as defined in Examples 1 to 4 (pH=10) by replacing the MTES formulation.

With the tests performed with an APTES formulation, process 3 or process 5 affords the hair a styling effect. The hair also better withstands washing.

1. A cosmetic process, comprising:
(i) contacting all or part of a surface of a keratin material with an effective amount of at least one emollient active agent selected from the group consisting of an ionic liquid based on guanidinium or dialkylimidazolium, a non-volatile or sparingly volatile oil, a wax, a thiol derivative, a phosphine, potassium hydroxide, ammonia, monoethanolamine, triethanolamine, calcium hydroxide, an alkaline salt of an amino acid, urea, a urea derivative, and a guanidine derivative,
(ii) contacting the surface with a cosmetic active agent, different from the emollient active agent, to be incorporated into the keratin at the surface of the keratin material, and
(iii) optionally heating the surface of the keratin material,
wherein (ii) and (iii) may each independently be performed prior to, simultaneously with or consecutive to (i).

2. The process of claim 1, in which the emollient active agent is at least one member selected from the group consisting of an ionic liquid based on guanidinium or dialkylimidazolium, cysteine, thioglycolic acid, and a guanidine derivative.

3. The process of claim 1, in which the emollient active agent comprises at least one guanidine derivative selected from the group consisting of guanidine hydroxide and guanidine carbonate.

4-5. (canceled)

6. The process of claim 1, in which the emollient active agent comprises the ionic liquid based on dialkylimidazolium, and the ionic liquid based on dialkylimidazolium is an ionic liquid based on a dialkylimidazolium acetate.

7. The process of claim 1, in which the emollient active agent comprises cysteine combined with guanidine carbonate.

8. The process of claim 1, in which the emollient active agent comprises tris(propionyl)phosphine.

9. The process of claim 1, in which the emollient active agent is a thiol derivative or a phosphine combined with a guanidinium-based ionic liquid.

10. (canceled)

11. The process of claim 1, in which the cosmetic active agent is a hardener or reinforcer.

12. (canceled)

13. The process of claim 11, in which the cosmetic active agent is a hardener that is an alkoxysilane compound.

14. The process of claim 1, in which the cosmetic active agent is at least one non-hardener.

15-19. (canceled)

20. The process of claim 1, in which the keratin materials are hair and/or nails.

21. A cosmetic process for hardening or reinforcing, in terms of thickness, a keratin material, comprising:
(i) contacting all or part of a surface of a keratin material with an effective amount of at least one emollient active agent that is capable of reducing by at least a factor of 4 a native Young's modulus of the keratin material; and
(ii) contacting the surface with a hardening or reinforcing active agent, different from the emollient active agent, to be incorporated into the keratin at the surface of the keratin material; and
(iii) optionally heating the surface of the keratin material,
wherein (ii) and (iii) may each independently be performed prior to, simultaneously with or consecutive to (i).

22. The process of claim 21, wherein the emollient active agent is selected from the group consisting of an ionic liquid based on guanidinium or dialkylimidazolium, a non-volatile or sparingly volatile oil, a wax, a thiol derivative, a phosphine, potassium hydroxide, ammonia, monoethanolamine, triethanolamine, calcium hydroxide, an alkaline salt of an amino acid, urea, a urea derivative, and a guanidine derivative.

23. The process of claim 21, in which the emollient active agent is at least one member selected from the group consisting of an ionic liquid based on guanidinium or dialkylimidazolium, cysteine, thioglycolic acid, and a guanidine derivative.

24. The process of claim 21, in which the keratin material is a keratin fiber, and the emollient active agent comprises 1-ethyl-3-methylimidazolium acetate.

25. The process of claim 21, in which the keratin material is a nail, and the emollient active agent comprises cysteine.

26. The process of claim 21, wherein the hardening or reinforcing agent is at least one member selected from the group consisting of an alkoxysilane compound, a siccative oil, a ceramide, a polymer and a crosslinking agent.

27. A cosmetic process for hardening nails, comprising:
(i) contacting all or part of a surface of a nail with an effective amount of at least one emollient active agent selected from the group consisting of an ionic liquid based on guanidinium or dialkylimidazolium, a thiol derivative, potassium hydroxide, ammonia, monoethanolamine, triethanolamine, calcium hydroxide, an alkaline salt of an amino acid, urea, a urea derivative, and a guanidine derivative,
(ii) contacting the surface with an alkoxysilane compound selected from the group consisting of (3-aminopropyl)triethoxysilane (APTES), methyltriethoxysilane (MTES) and octyltriethoxysilane (OTES), to be incorporated into the keratin at the surface of the nail, and
(iii) optionally heating the surface of the nail,
wherein (ii) and (iii) may each independently be performed prior to, simultaneously with or consecutive to (i).

28. The process of claim 27, in which the emollient active agent comprises at least one guanidine derivative selected from the group consisting of guanidine hydroxide and guanidine carbonate.

29. (canceled)

30. The process of claim 27, in which the emollient active agent comprises cysteine or a cysteine/guanidine carbonate mixture.
